(19) United States
(12) Patent Application Publication
(10) Pub. No.: US 2023/0411014 A1
(43) Pub. Date: Dec. 21, 2023

(54) APPARATUS AND METHOD FOR TRAINING OF MACHINE LEARNING MODELS USING ANNOTATED IMAGE DATA FOR PATHOLOGY IMAGING

(71) Applicant: Leica Biosystems Imaging, Inc., Vista, CA (US)

(72) Inventor: Allen H. OLSON, Vista, CA (US)

(21) Appl. No.: 18/459,679

(22) Filed: Sep. 1, 2023

Related U.S. Application Data
(63) Continuation of application No. PCT/US2022/020738, filed on Mar. 17, 2022.
(60) Provisional application No. 63/162,698, filed on Mar. 18, 2021.

(51) Int. Cl. G16H 50/20; G16H 30/40

(52) U.S. Cl. CPC G16H 50/20; G16H 30/40

(57) ABSTRACT

Features are disclosed for training a machine learning model to identify objects in histological images. A system may obtain an image and determine a number of objects in the image. For example, the system may determine a percentage of objects in the image with a particular object type. Further, the system may determine a weight. The weight may specify a percentage of the image occupied by objects with the particular object type. The system can generate training set data that includes the image, data identifying the number of objects in the image, and the weight. The system can use the training set data to train a machine learning model to predict a number of objects in a different image and a weight. The system can implement the machine learning model based on training the machine learning model.

### RELATED APPLICATION(S)

This application claims the benefit of priority of U.S. Provisional Patent Application No. 63/162,698, filed Mar. 18, 2021, entitled IMPROVED ANNOTATION METHOD AND SYSTEM FOR TRAINING OF MACHINE LEARNING MODELS IN PATHOLOGY IMAGING, which is incorporated herein by reference in its entirety.

### BACKGROUND

### Technical Field

The described technology relates to histology, and in particular, techniques for training machine learning models for pathology imaging.

### Description of the Related Technology

Tissue samples can be analyzed under an image analysis system for various diagnostic purposes, including detecting cancer by identifying structural abnormalities in the tissue sample. A tissue sample can be imaged to produce image data using the image analysis system. The image analysis system can capture the image data and perform the visual image analysis on the image data to determine particular image characteristics of an image of the tissue sample. Visual image analysis can aid in medical diagnosis and examination.

### SUMMARY

One aspect of the present disclosure is an apparatus. The apparatus can include a memory circuit storing computer-executable instructions and a hardware processing unit configured to execute the computer-executable instructions. The hardware processing unit can obtain a first slide image comprising a first plurality of objects. Further, the hardware processing unit can determine a number of the first plurality of objects in the first slide image and a first weight. Further, the hardware processing unit can generate training set data that includes the first slide image, object data identifying the number of the first plurality of objects in the first slide image, and weight data identifying the first weight. Further, the hardware processing unit can train a machine learning model based on the training set data. The hardware processing unit can implement the machine learning model. The machine learning model can predict a number of a second plurality of objects in a second slide image and a second weight.

In another aspect of the present disclosure, the hardware processing unit can obtain, from memory, the first slide image. Further, the hardware processing unit can obtain, from a user computing device, user input identifying the number of the first plurality of objects in the first slide image.

In another aspect of the present disclosure, the hardware processing unit can cause display, via a display of a user computing device, of the first slide image. Further, the hardware processing unit can obtain, from the user computing device, user input identifying the number of the first plurality of objects in the first slide image based on causing display of first slide image.

In another aspect of the present disclosure, the machine learning model may include a convolutional neural network.

In another aspect of the present disclosure, the first slide image may correspond to a portion of an image. The number of the first plurality of objects in the first slide image may include a number of the first plurality of objects in the portion of the image. The hardware processing unit can obtain, from a user computing device, user input identifying the portion of the image. The training data set further may include the portion of the image.

In another aspect of the present disclosure, the number of the first plurality of objects in the first slide image may include a number of the first plurality of objects in a portion of an image. The hardware processing unit can obtain, from a user computing device, first user input identifying the portion of the image. Further, the hardware processing unit can obtain, from the user computing device, second user input identifying the number of the first plurality of objects in the first slide image.

In another aspect of the present disclosure, the first slide image may correspond to a portion of an image. The number of the first plurality of objects in the first slide image may include a ratio of a count of objects in the first slide image to a count of objects in the image.

In another aspect of the present disclosure, the first plurality of objects may include at least one of invasive cells, invasive cancer cells, in-situ cancer cells, lymphocytes, stroma, abnormal cells, normal cells, or background cells.

In another aspect of the present disclosure, the hardware processing unit can obtain a third slide image including a third plurality of objects. Further, the hardware processing unit can determine a number of the third plurality of objects in the third slide image and a third weight. The training set data may include the third slide image, additional object data identifying the number of the third plurality of objects in the third slide image, and additional weight data identifying the third weight.

In another aspect of the present disclosure, the first slide image may correspond to a first portion of an image. Further, the hardware processing unit can obtain a third slide image corresponding to a second portion of the image. The third slide image may include a third plurality of objects. Further, the hardware processing unit can determine a number of the third plurality of objects in the third slide image and a third weight. The first weight may be based on an amount of the first portion of the image occupied by the first plurality of objects and the third weight may be based on an amount of the second portion of the image occupied by the third plurality of objects. The training set data may include the third weight, the third slide image, and additional object data identifying the number of the third plurality of objects in the third slide image.

In another aspect of the present disclosure, the machine learning model further can predict a number of a third plurality of objects in a third slide image. The second slide image may correspond to a first portion of an image and the third slide image may correspond to a second portion of the image. Further, the hardware processing unit can train a second machine learning model based on the number of the second plurality of objects in the second slide image and the number of the third plurality of objects in the third slide image. Further, the hardware processing unit can implement the second machine learning model. The second machine learning model may aggregate a plurality of predictions for a plurality of slide images to identify a number of a plurality of objects in an image. Each of the plurality of predictions can identify a number of a plurality of objects in a corresponding slide image of the plurality of slide images.

In another aspect of the present disclosure, the first plurality of objects may correspond to a particular object type of a plurality of object types.

Another aspect of the present disclosure is a method including obtaining a first slide image comprising a first plurality of objects. The method may further include determining a number of the first plurality of objects in the first slide image and a first weight. The method may further include generating training set data including the first slide image, object data identifying the number of the first plurality of objects in the first slide image, and weight data identifying the first weight. The method may further include training a machine learning model based on the training set data. The method may further include implementing the machine learning model. The machine learning model may predict a number of a second plurality of objects in a second slide image and a second weight.

Another aspect of the present disclosure is a non-transitory computer-readable medium storing computer-executable instructions that may be executed by one or more computing devices. The one or more computing devices may obtain a first slide image comprising a first plurality of objects. Further, the one or more computing devices can determine a number of the first plurality of objects in the first slide image and a first weight. Further, the one or more computing devices can generate training set data including the first slide image, object data identifying the number of the first plurality of objects in the first slide image, and weight data identifying the first weight. Further, the one or more computing devices can train a machine learning model based on the training set data. Further, the one or more computing devices can implement the machine learning model. The machine learning model can predict a number of a second plurality of objects in a second slide image and a second weight.

In another aspect of the present disclosure, the one or more computing devices can obtain, from a user computing device, user input identifying the number of the first plurality of objects in the first slide image.

In another aspect of the present disclosure, the first slide image may correspond to a portion of an image. The number of the first plurality of objects in the first slide image may include a percentage of the number of the first plurality of objects in the first slide image as compared to a number of a plurality of objects in the image.

In another aspect of the present disclosure, the first plurality of objects can include at least one of invasive cells, invasive cancer cells, in-situ cancer cells, lymphocytes, stroma, abnormal cells, normal cells, or background cells.

In another aspect of the present disclosure, the one or more computing devices can obtain a third slide image comprising a third plurality of objects. Further, the one or more computing devices can determine a number of the third plurality of objects in the third slide image and a third weight. The training set data further may include the third slide image, additional object data identifying the number of the third plurality of objects in the third slide image, and additional weight data identifying the third weight.

In another aspect of the present disclosure, the first slide image may correspond to a first portion of an image. The first weight may be based on an amount of the first portion of the image occupied by the first plurality of objects.

In another aspect of the present disclosure, the machine learning model cam further predict a number of a third plurality of objects in a third slide image. The second slide image can correspond to a first portion of an image and the third slide image can correspond to a second portion of the image. The one or more computing devices can train a second machine learning model based on the number of the second plurality of objects in the second slide image and the number of the third plurality of objects in the third slide image. Further, the one or more computing devices can implement the second machine learning model. The second machine learning model can aggregate a plurality of predictions for a plurality of slide images to identify a number of a plurality of objects in an image. Each of the plurality of predictions can identify a number of a plurality of objects in a corresponding slide image of the plurality of slide images.

Another aspect of the present disclosure is an apparatus including a memory circuit storing computer-executable instructions indicative of a prediction model that identifies tumorous cells and a hardware processing unit configured to execute the computer-executable instructions to implement the prediction model to identify the tumorous cells. The prediction model can predict a number of a second plurality of objects in a second slide image and a second weight. The prediction model may be characterized by a training of the prediction model that may include obtaining a first slide image comprising a first plurality of objects, determining a number of the first plurality of objects in the first slide image and a first weight, generating training set data including the first slide image, object data identifying the number of the first plurality of objects in the first slide image, and weight data identifying the first weight, and training the prediction model based on the training set data.

Another aspect of the present is a method for training a machine learning model by a server. The method may include obtaining, by the server, a first slide image including a first plurality of objects. The method may further include determining, by the server, a number of the first plurality of objects in the first slide image and a first weight. Further, the method may include generating, by the server, training set data including the first slide image, object data identifying the number of the first plurality of objects in the first slide image, and weight data identifying the first weight. Further, the method may include training, by the server, the machine learning model based on the training set data. Further, the method may include implementing, by the server, the machine learning model. The machine learning model can predict a number of a second plurality of objects in a second slide image and a second weight.

### BRIEF DESCRIPTION OF THE DRAWINGS

The features and advantages of the multi-stage stop devices, systems, and methods described herein will become more fully apparent from the following description and appended claims, taken in conjunction with the accompanying drawings. These drawings depict several embodiments in accordance with the disclosure and are not to be considered limiting of its scope. In the drawings, similar reference numbers or symbols typically identify similar components, unless context dictates otherwise. The drawings may not be drawn to scale.

FIG. 1 illustrates an example environment in which a user and/or an imaging system may implement an image analysis system according to some embodiments.

FIG. 2 depicts an example workflow for generating image data from a tissue sample block according to some embodiments.

FIG. 3A illustrates an example prepared tissue block according to some embodiments.

FIG. 3B illustrates an example prepared tissue block and an example prepared tissue slice according to some embodiments.

FIG. 4 shows an example imaging device, according to one embodiment.

FIG. 5 is an example computing system which can implement any one or more imaging devices, image analysis system, and user computing device of the multispectral imaging system illustrated in FIG. 1.

FIG. 6 depicts a schematic diagram of a machine learning algorithm, including a multiple layer neural network in accordance with aspects of the present disclosure.

FIG. 7A is a schematic drawing of a neural network architecture used in on embodiment of the invention.

FIG. 7B illustrates a combination of global and local feature maps to generate a feature map within the neural network architecture of FIG. 7A.

FIG. 8 is a flowchart of an example routine for training a machine learning model using training data.

FIG. 9 is a flowchart of an example routine for generating predictions using a machine learning model.

FIG. 10 is a block diagram of a TPU which may be used for performing the computations involved in implementing the neural network architecture of FIGS. 7A and 7B.

FIG. 11 illustrates an example computer network which can be used in conjunction with embodiments of this invention.

FIG. 12A illustrates an example image and an example numerical representation of objects in the example image.

FIG. 12B illustrates an example image and an example numerical representation of objects in the example image.

FIG. 12C illustrates an example image and an example numerical representation of objects in the example image.

FIG. 12D illustrates an example image and an example numerical representation of objects in the example image.

FIG. 13A is a flowchart of an example routine for generating training data for training a machine learning model.

FIG. 13B is a flowchart of an example routine for training a machine learning model and providing output of the machine learning model to another machine learning model.

### DETAILED DESCRIPTION

Generally described, the present disclosure relates to an image analysis system that can receive an image (e.g., a slide image) of a histological sample (e.g., a tissue block) and determine a number of objects in the image (e.g., a percentage of objects, a quantity of objects, etc.). The image analysis system can determine the number of objects in the image and can perform various operations based on the identified number of objects, such as outputting the number of objects for display via a user computing device.

In order to identify objects within a slide image, the image analysis system can implement and/or can include an image analysis module (e.g., a convolutional neural network, a machine learning algorithm, a machine learning model, etc.) that analyzes each image. As described herein, the use of an image analysis module within such an image analysis system can increase the efficiency of the imaging process. Specifically, by training the image analysis module to identify a number of objects in the image using a training data set, the efficiency of the training of the image analysis module and the efficiency of the imaging process can be increased. For example, the image analysis module can be trained to identify the number of objects in the image with less training data than an image analysis module that is trained to identify the outlines of objects in an image.

As used herein, the term “image analysis system” may refer to any electronic device or component(s) capable of performing an image analysis process. For example, an “image analysis system” may comprise a scanner, a camera, etc. In some embodiments, the image analysis system may not perform the imaging and, instead, may receive the image data and perform image analysis on the image data.

As described herein, an image analysis system can be used to perform image analysis on received image data (e.g., image data corresponding to histological samples). The image analysis system can obtain (e.g., via imaging performed by the image analysis system or via imaging performed by an imaging device) image data of a first histological sample and image data of a second histological sample. Each histological sample can be associated with a particular tissue block and/or a section of a particular tissue block, and the image analysis system may implement the image analysis module to identify objects within the image data. Specifically, the image analysis system may implement the image analysis module to identify cancerous cells within the image data. The image analysis system may train the image analysis module to identify the cancerous cells using a training data set.

In some cases, the image analysis system may train the image analysis module using a training data set that indicates the objects in training image data. For example, the training data set may indicate that the training image data includes a first cancerous cell. This may be sufficient where the training image data includes a single cell. However, such a training data set may not provide satisfactory results in particular circumstances or for particular users. For example, the training image data may include multiple cells and an indication that the training image data includes a first cancerous cell may not be sufficient to identify which of the multiple cells is the first cancerous cell. Image data may include a plurality of objects that each corresponds to a different object size, a different object type, etc. Further, the image data may include a plurality of objects that are intermixed or dispersed across a Field of View (FOV), Due to the intermixing of the plurality of objects across the FOV, the image data may not be efficiently separated into sub-images that each correspond to a single object and/or a single object type. Therefore, training the image analysis module using a training data set that indicates cells located in the training image data may not be sufficient.

In some cases, the image analysis system may train the image analysis module using a training data set that includes outlines within the training image data for each object in the training image data. For example, the training data set may identify an outline and a label associated with the outline (e.g., a cancerous cell). However, the generation of the outlines may be a time consuming and inefficient process. For example, the image analysis system (or a system separate from the image analysis system) may provide image data to a user computing device and/or cause display of the image data at a user computing device. The image analysis system may provide the image data to the user computing device for outlining (e.g., via a drawing) by a user. Further, the image analysis system may generate training image data based on the outline by the user (e.g., a hand drawn outline). Such an outline by the user may be provided using a user interface of a user computing device (with imaging and outlining capabilities). Due to the complexity of the image data (and the complexity of pathology images in general), the outlining of image data by a user may be inefficient and time consuming. Further, the outlining of image data by a user may rely on outlining of the image data by a trained pathologist (e.g., a trained pathologist with particular subject matter expertise). In some cases, the outlining of the image data by the trained pathologist may be an expensive, time consuming process. For example, the trained pathologist may require additional training to perform the outlining of the image data.

In some cases, the image analysis system may require a large training data set to train the image analysis module to accurately identify objects in image data (e.g., a training data set corresponding to 100, 500, 1,000, etc. images). As the generation of the training data set may be based on a user computing device providing outlines of the image data by a user, the generation of a large training data set may be a time consuming and inefficient process.

In many conventional cases, implementing a generic image analysis system to perform the image analysis process may not provide satisfactory results in particular circumstances or for particular users. Such generic image analysis system s may determine that images of histological samples include particular objects based on a user input. For example, the image analysis system may receive a generic training data set that includes one or more outlines (e.g., generated by a user) of one or more objects in the training image data. Such a generic image analysis system may cause objects to be erroneously identified based on user input identifying the outlines. For example, if the user is not a trained pathologist with particular subject matter expertise, the outlines may erroneously identify one or more objects, one or more object sizes, one or more object types, etc. Due to the user error, the generic image analysis system may be trained erroneously to identify objects within received image data. As the image data corresponds to histological samples (e.g., tissue blocks), slices of histological samples, or other tissue samples, it can be crucial to identify objects within the image data. An erroneous identification of an object within the image data and/or a failure to identify objects within the image data can result in misdiagnosis. Such a misdiagnosis can lead to additional adverse consequences. Further, by requiring such extensive user input that includes outlines of the objects within image data, the training process and/or the imaging process can result in performance issues. For example, the training process for a generic image analysis system may be slow, inefficient, and non-effective. Conventional image analysis systems may therefore be inadequate in the aforementioned situations.

As image analysis systems proliferate, the demand for faster and more efficient image processing and training of image analysis systems has also increased. The present disclosure provides a system for training an image analysis module with significant advantages over prior implementations. The present disclosure provides systems and methods that enable an increase in the speed and efficiency of the training process for the image analysis system, relative to traditional image analysis systems without significantly affecting the accuracy of the image analysis system. These advantages are provided by the embodiments discussed herein, and specifically by the implementation of an image analysis module that is trained using a training data set that indicates a number of objects in training image data. Further, the use of an image analysis module that is trained using a training data set enables the image analysis module to be trained without outlines provided by a user, thereby increasing the efficiency and speed of the training process according to the above methods.

Some aspects of this disclosure relate to training an image analysis module (e.g., a machine learning algorithm) for image classification and/or segmentation. The image analysis system described herein can provide improved efficiency and speed based on training an image analysis module using a training data set that identifies a number of objects in the image. Therefore, the image analysis module can be trained to identify a number of objects in an image using the training data set. An image analysis module that is trained using such a training data set is able to provide a training process with increased efficiency and speed without significantly affecting the capabilities or utility of the image analysis module. Specifically, a user may not require an outline of each object within an image. Instead, a user may require an image analysis module that identifies a number of objects in an image. Such an identification of the number of objects may be sufficient for the user (e.g., a trained pathologist) to identify the objects in the image.

The image analysis system may request a training data set for training the image analysis module. For example, the image analysis system may request a user computing device to provide the training data set. Specifically, the image analysis system may provide image data to the user computing device and request the generation of training image data using the image data. In some cases, the user computing device may identify the image data and/or may obtain the image data from a different computing system.

Based on the request for the training data set, the user computing device may obtain image data. Further, the user computing device may obtain data identifying a portion of the image data (e.g., a FOV). In some embodiments, the data may identify all of the image data. The data identifying the portion of the image data may include a particular shape. For example, the data identifying the portion of the image data may include a rectangle, a circle, an oval, a square, a triangle, or any other shape. Further, the data identifying the portion of the image data may include a regularly shaped area and/or an irregularly shaped area. In some cases, the shape may be drawn by a user (e.g., hand drawn). For example, the user may draw the shape via a touch screen of the user computing device.

The user computing device may further obtain a number of objects in the image data. The user may provide the number of objects in the image data as input to the user computing device. The number of objects in the image data may specify the percentage of objects in the image data that are located in the portion of the image data (e.g., the ratio of objects located in the portion of the image data to the objects located in the image data). Specifically, the image analysis system may determine the number of objects in the image data by dividing the number of objects in a portion of the image by the total number of objects in the image. For example, the image data may include 100 objects and the portion of the image data may include 25 objects, therefore, the number of objects in the image data may be 25%. In another example, the portion of the image data may include all of the image data and the number of objects in the image data may be 100%.

In some cases, the number of objects in the image data may specify a numerical count of the number of objects located in the portion of the image data. For example, the image data may include 100 objects and the portion of the image data may include 25 objects, therefore, the number of objects in the image data may be 25.

In some cases, the number of objects in the image data may specify a phrase, a symbolical representation, etc. that represents the number of objects located in the portion of the image data. For example, the image data may include 100 objects and the portion of the image data may include 25 objects, therefore, the number of objects in the image data may be “low” or “−.” It will be understood that the number of objects in the image data may include any numerical, alphabetical, alphanumerical, symbolical, etc. representation of the number of objects in the image data.

Further, the image analysis system may determine a weight for the portion of the image data. The weight may identify an amount of the particular portion of the image data occupied by the objects. For example, an amount of the particular portion of the image data occupied by the objects may specify the percentage of the portion of the image data occupied by objects (e.g., the ratio of the size of the portion of the image data to the size of the image data (within the portion of the image data) occupied by objects). Specifically, the image analysis system may determine the amount of the particular portion of the image data occupied by the objects by dividing the area of an image occupied by objects by the total area of the image. For example, the portion of the image data may include 100 square millimeters and the 25 square millimeters of the portion of the image data may include objects, therefore, the weight may be 25%.

As described herein, the image analysis system may obtain training image data that identifies the portion of the image data, the number of objects located in the portion of the image data, and the weight. The image analysis system may generate a training data set using the training image data. Further, the image analysis system may train the image analysis module, using the training data set, to predict a number of objects located in an additional image (e.g., a portion of an additional image) and a corresponding weight. Based on the training by the image analysis system, the image analysis module can obtain data identifying a particular portion of image data (e.g., a FOV) and identify (e.g., predict) the number of objects in the specified portion of image data and a weight for the specified portion of image data.

In some cases, the image analysis system may obtain a plurality of predictions by the image analysis module that identifies a plurality of portions of image data (e.g., a plurality of portions of image data corresponding to a single image), a corresponding plurality of numbers of objects located in the plurality of portions of image data, and a corresponding plurality of weights. The image analysis system may generate a combined prediction (e.g., a prediction for a single image that include the plurality of portions of image data) by aggregating the plurality of predictions (e.g., based on a weighted average). For example, for each portion of image data, the image analysis system may multiple the number of objects located in the image data by a corresponding weight to determine a weighted number for the corresponding portion of image data. Further, the image analysis system may aggregate each of the weighted numbers. The image analysis system may aggregate each of the weights and divide the aggregated, weighted numbers by the aggregated weights to determine the weighted average.

In some cases, the image analysis system may aggregate the plurality of predictions using a second image analysis module (e.g., a second machine learning algorithm). The image analysis system may obtain output from the image analysis module (e.g., a first image analysis module) identifying the plurality of portions of image data (e.g., a plurality of portions of image data corresponding to a single image), a corresponding plurality of numbers of objects located in the plurality of portions of image data, and a corresponding plurality of weights. Based on the obtained output, the second image analysis module may identify (e.g., predict) the number of objects in multiple portions of image data (e.g., a single image).

The image analysis system may train the second image analysis module using an additional training data set that specifies multiple portions of image data, a number of objects located in each portion of the image data, and a corresponding weight for each portion of the image data. Further, the second image analysis module may be trained to identify (e.g. predict) a weight for the multiple portions of image data. In some cases, the image analysis module and the second image analysis module may be combined into a single image analysis module.

The features of the systems and methods for machine learning model training in the context of pathology imaging will now be described in detail with reference to certain embodiments illustrated in the figures. The illustrated embodiments described herein are provided by way of illustration and are not meant to be limiting. Other embodiments can be utilized, and other changes can be made, without departing from the spirit or scope of the subject matter presented. It will be readily understood that the aspects and features of the present disclosure described below and illustrated in the figures can be arranged, substituted, combined, and designed in a wide variety of different configurations by a person of ordinary skill in the art, all of which are made part of this disclosure.

### Tissue Sample Overview

As discussed above, the image analysis module may be utilized in the diagnosis of tissue samples. The diagnosis of tissue samples may involve several processing steps to prepare the tissue sample for viewing under a microscope. While traditional diagnostics techniques may involve staining a tissue sample to provide additional visual contrast to the cellular structure of the sample when viewed under a microscope and manually diagnosing a disease by viewing the stained image through the microscope, optical scanning on the sample can be used to create image data which can be “virtually” stained using an image analysis system and provided to an image analysis system for processing. In certain implementations, the optical scanning may be performed using multispectral imaging (also referred to as multispectral optical scanning) to provide additional information compared to optical scanning using a single frequency of light. As discussed above, in some implementations, the image analysis system can include a machine learning algorithm trained to identify and diagnose one or more diseases by identifying structures or features present in the image data that are consistent with training data used to train the machine learning algorithm.

Multispectral imaging may involve providing multispectral light to the tissue sample using a multispectral light source and detecting light emitted from the sample in response to the multispectral light using an imaging sensor. Under certain wavelengths/frequencies of the multispectral light, the tissue sample may exhibit autofluorescence which can be detected to generate image data that can be virtually stained. The use of virtual staining of tissue samples may enable various improvements in the histology workflow. For example, image data produced during virtual staining can be provided to a machine learning algorithm (also referred to as an artificial intelligence “AI” algorithm) which can be trained to provide a diagnosis of a disease present in the tissue sample.

However, there may be limitations to the data that can be obtained using only virtual staining. That is, while virtual staining may be able to produce markers that are substantially similar to certain chemical stains (e.g., hematoxylin and eosin (H&E) stains), markers which are produced using other chemical stains (e.g., immunohistochemistry (IHC) stains) may not be easily achieved using virtual staining. Thus, it may still be necessary to apply chemical stains to a tissue sample in order to fully diagnose a disease.

As used herein, chemical staining generally refers to the physical staining of a tissue sample using an assay in order to provide additional visual contrast to certain aspects of the cellular structure of the tissue sample. There are at least three there common types of chemical stains that are used in addition to H&E staining. Any one or more of the below example types of chemical stains, or other types of chemical stains not explicitly listed below, may be used in accordance with aspects of this disclosure.

The first type of chemical stain is termed a “special stain,” which typically involves washing one or more chemical dyes the tissue sample in order to highlight certain features of interest (e.g., bacteria and/or fungi) or to enable contrast for viewing of cell morphology and/or tissue structures (e.g., highlighting carbohydrate deposits).

The second type of chemical stain is termed immunohistochemistry (IHC), and typically involves using antibody markers to identify particular proteins within the tissue sample. These antibodies can be highlighted using visible, fluorescent, and/or other detection methods.

The third type of chemical stain may be termed molecular testing (e.g., in situ hybridization (ISH)), and typically involves using an assay to identify specific DNA or RNA mutations in the genome. These mutations can also be highlighted using visible, fluorescent, and/or other detection methods.

With traditional histology workflow, the total length of time between a tissue biopsy and the time at which a pathologist is able to determine the final diagnosis of a disease present in the tissue sample is typically greater than the length of time between a virtual staining and a final diagnosis. For example, traditional histology may involve first obtaining the tissue sample (e.g., via a biopsy) and performing an initial stain on at least one slice of the tissue sample (e.g., an H&E stain) at a lab. After the initial stain, the remainder of the tissue sample from which the slice was obtained is typically stored to preserve the tissue sample for further staining. Storing the tissue sample and retrieving the stored tissue sample for chemical staining may involve additional steps performed at the lab, increasing the length of time between the tissue biopsy and the final diagnosis.

The lab can produce one or more images based on the stained tissue sample which are typically sent to the pathologist at the end of the day. The pathologist reviews the image of the stained slide, and based on an initial diagnosis of the slide, may order one or more other chemical stains to aid in the diagnosis. The lab receives the orders, retrieves the stored tissue sample, and performs the ordered chemical stains on new slices of the tissue sample, and sends the subsequent stained slides to the pathologist. In other implementations, digital images of the stained slides may be sent to the pathologist in addition to or in place of the physical slides. After receiving the slides/images, the pathologist can complete the diagnosis using the images produced based on both sets of stained slides. However, it can be difficult for the pathologist to mentally matching similar features on different sections/slides because the features may be aligned differently due to the necessity of staining separate slices of the tissue sample.

Although the total length of active time involved in the histological workflow may be less than about 24 hours, due to the downtime associated with transmitting images between the lab and the pathologist, along with scheduling the time of the lab technician and the pathologist, the amount of real time elapsed between taking the biopsy and final diagnosis range from about one week for simple cases to about 50 days on average or longer for more complex diagnoses. It is desirable to reduce the time between taking the biopsy and the final diagnosis without significantly altering the scheduling demands on the lab technician or the pathologist.

Aspects of this disclosure relate to systems and methods for hybrid virtual and chemical staining of tissue samples which can address one or more of the issues relating to timing and workflow. Advantageously, aspects of this disclosure can use both virtual and chemical staining in the histology workflow, which may significantly reduce the amount of time required to arrive at the final diagnosis.

### System Overview

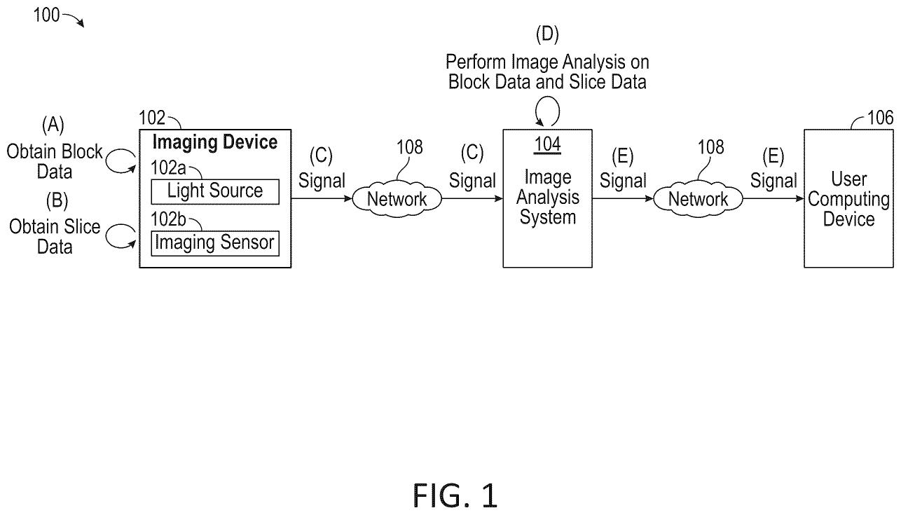

FIG. 1 illustrates an example environment 100 (e.g., a hybrid virtual and chemical staining system) in which a user and/or the multispectral imaging system may implement an image analysis system 104 according to some embodiments. The image analysis system 104 may perform image analysis on received image data. The image analysis system 104 can perform virtual staining on the image data obtained using multispectral imaging for input to a machine learning algorithm. Based on image data generated during virtual staining, the machine learning algorithm can generate a first diagnosis which may include an indication of whether the image data is indicative of a disease present in the tissue sample.

The image analysis system 104 may perform the image analysis using an image analysis module (not shown in FIG. 1). The image analysis system 104 may receive the image data from an imaging device 102 and transmit the recommendation to a user computing device 106 for processing. Although some examples herein refer to a specific type of device as being the imaging device 102, the image analysis system 104, or the user computing device 106, the examples are illustrative only and are not intended to be limiting, required, or exhaustive. The image analysis system 104 may be any type of computing device (e.g., a server, a node, a router, a network host, etc.). Further, the imaging device 102 may be any type of imaging device (e.g., a camera, a scanner, a mobile device, a laptop, etc.). In some embodiments, the imaging device 102 may include a plurality of imaging devices. Further, the user computing device 106 may be any type of computing device (e.g., a mobile device, a laptop, etc.).

In some implementations, the imaging device 102 includes a light source 102a configured to emit multispectral light onto the tissue sample(s) and the image sensor 102b configured to detect multispectral light emitted from the tissue sample. The multispectral imaging using the light source 102a can involve providing light to the tissue sample carried by a carrier within a range of frequencies. That is, the light source 102a may be configured to generate light across a spectrum of frequencies to provide multispectral imaging.

In certain embodiments, the tissue sample may reflect light received from the light source 102a, which can then be detected at the image sensor 102b. In these implementations, the light source 102a and the image sensor 102b may be located on substantially the same side of the tissue sample. In other implementations, the light source 102a and the image sensor 102b may be located on opposing sides of the tissue sample. The image sensor 102b may be further configured to generate image data based on the multispectral light detected at the image sensor 102b. In certain implementations, the image sensor 102b may include a high-resolution sensor configured to generate a high-resolution image of the tissue sample. The high-resolution image may be generated based on excitation of the tissue sample in response to laser light emitted onto the sample at different frequencies (e.g., a frequency spectrum).

The imaging device 102 may capture and/or generate image data for analysis. The imaging device 102 may include one or more of a lenses, an image sensor, a processor, or memory. The imaging device 102 may receive a user interaction. The user interaction may be a request to capture image data. Based on the user interaction, the imaging device 102 may capture image data. In some embodiments, the imaging device 102 may capture image data periodically (e.g., every 10, 20, or 30 minutes). In other embodiments, the imaging device 102 may determine that an item has been placed in view of the imaging device 102 (e.g., a histological sample has been placed on a table and/or platform associated with the imaging device 102) and, based on this determination, capture image data corresponding to the item. The imaging device 102 may further receive image data from additional imaging devices. For example, the imaging device 102 may be a node that routes image data from other imaging devices to the image analysis system 104. In some embodiments, the imaging device 102 may be located within the image analysis system 104. For example, the imaging device 102 may be a component of the image analysis system 104. Further, the image analysis system 104 may perform an imaging function. In other embodiments, the imaging device 102 and the image analysis system 104 may be connected (e.g., wirelessly or wired connection). For example, the imaging device 102 and the image analysis system 104 may communicate over a network 108. Further, the imaging device 102 and the image analysis system 104 may communicate over a wired connection. In one embodiment, the image analysis system 104 may include a docking station that enables the imaging device 102 to dock with the image analysis system 104. An electrical contact of the image analysis system 104 may connect with an electrical contact of the imaging device 102. The image analysis system 104 may be configured to determine when the imaging device 102 has been connected with the image analysis system 104 based at least in part on the electrical contacts of the image analysis system 104. In some embodiments, the image analysis system 104 may use one or more other sensors (e.g., a proximity sensor) to determine that an imaging device 102 has been connected to the image analysis system 104. In some embodiments, the image analysis system 104 may be connected to (via a wired or a wireless connection) a plurality of imaging devices.

The image analysis system 104 may include various components for providing the features described herein. In some embodiments, the image analysis system 104 may include one or more image analysis modules to perform the image analysis of the image data received from the imaging device 102. The image analysis modules may perform one or more imaging algorithms using the image data.

The image analysis system 104 may be connected to the user computing device 106. The image analysis system 104 may be connected (via a wireless or wired connection) to the user computing device 106 to provide a recommendation for a set of image data. The image analysis system 104 may transmit the recommendation to the user computing device 106 via the network 108. In some embodiments, the image analysis system 104 and the user computing device 106 may be configured for connection such that the user computing device 106 can engage and disengage with image analysis system 104 in order to receive the recommendation. For example, the user computing device 106 may engage with the image analysis system 104 upon determining that the image analysis system 104 has generated a recommendation for the user computing device 106. Further, a particular user computing device 106 may connect to the image analysis system 104 based on the image analysis system 104 performing image analysis on image data that corresponds to the particular user computing device 106. For example, a user may be associated with a plurality of histological samples. Upon determining, that a particular histological sample is associated with a particular user and a corresponding user computing device 106, the image analysis system 104 can transmit a recommendation for the histological sample to the particular user computing device 106. In some embodiments, the user computing device 106 may dock with the image analysis system 104 in order to receive the recommendation.

In some implementations, the imaging device 102, the image analysis system 104, and/or the user computing device 106 may be in wireless communication. For example, the imaging device 102, the image analysis system 104, and/or the user computing device 106 may communicate over a network 108. The network 108 may include any viable communication technology, such as wired and/or wireless modalities and/or technologies. The network may include any combination of Personal Area Networks (“PANs”), Local Area Networks (“LANs”), Campus Area Networks (“CANs”), Metropolitan Area Networks (“MANs”), extranets, intranets, the Internet, short-range wireless communication networks (e.g., ZigBee, Bluetooth, etc.), Wide Area Networks (“WANs”)—both centralized and/or distributed—and/or any combination, permutation, and/or aggregation thereof. The network 108 may include, and/or may or may not have access to and/or from, the internet. The imaging device 102 and the image analysis system 104 may communicate image data. For example, the imaging device 102 may communicate image data associated with a histological sample to the image analysis system 104 via the network 108 for analysis. The image analysis system 104 and the user computing device 106 may communicate a recommendation corresponding to the image data. For example, the image analysis system 104 may communicate a diagnosis regarding whether the image data is indicative of a disease present in the tissue sample based on the results of a machine learning algorithm. In some embodiments, the imaging device 102 and the image analysis system 104 may communicate via a first network and the image analysis system 104 and the user computing device 106 may communicate via a second network. In other embodiments, the imaging device 102, the image analysis system 104, and the user computing device 106 may communicate over the same network.

With reference to an illustrative embodiment, at [A], the imaging device 102 can obtain block data. In order to obtain the block data, the imaging device 102 can image (e.g., scan, capture, record, etc.) a tissue block. The tissue block may be a histological sample. For example, the tissue block may be a block of biological tissue that has been removed and prepared for analysis. As will be discussed in further below, in order to prepare the tissue block for analysis, various histological techniques may be performed on the tissue block. The imaging device 102 can capture an image of the tissue block and store corresponding block data in the imaging device 102. The imaging device 102 may obtain the block data based on a user interaction. For example, a user may provide an input through a user interface (e.g., a graphical user interface (“GUI”)) and request that the imaging device 102 image the tissue block. Further, the user can interact with imaging device 102 to cause the imaging device 102 to image the tissue block. For example, the user can toggle a switch of the imaging device 102, push a button of the imaging device 102, provide a voice command to the imaging device 102, or otherwise interact with the imaging device 102 to cause the imaging device 102 to image the tissue block. In some embodiments, the imaging device 102 may image the tissue block based on detecting, by the imaging device 102, that a tissue block has been placed in a viewport of the imaging device 102. For example, the imaging device 102 may determine that a tissue block has been placed on a viewport of the imaging device 102 and, based on this determination, image the tissue block.

At [B], the imaging device 102 can obtain slice data. In some embodiments, the imaging device 102 can obtain the slice data and the block data. In other embodiments, a first imaging device can obtain the slice and a second imaging device can obtain the block data. In order to obtain the slice data, the imaging device 102 can image (e.g., scan, capture, record, etc.) a slice of the tissue block. The slice of the tissue block may be a slice of the histological sample. For example, the tissue block may be sliced (e.g., sectioned) in order to generate one or more slices of the tissue block. In some embodiments, a portion of the tissue block may be sliced to generate a slice of the tissue block such that a first portion of the tissue block corresponds to the tissue block imaged to obtain the block data and a second portion of the tissue block corresponds to the slice of the tissue block imaged to obtain the slice data. As will be discussed in further detail below, various histological techniques may be performed on the tissue block in order to generate the slice of the tissue block. The imaging device 102 can capture an image of the slice and store corresponding slice data in the imaging device 102. The imaging device 102 may obtain the slice data based on a user interaction. For example, a user may provide an input through a user interface and request that the imaging device 102 image the slice. Further, the user can interact with imaging device 102 to cause the imaging device 102 to image the slice. In some embodiments, the imaging device 102 may image the tissue block based on detecting, by the imaging device 102, that the tissue block has been sliced or that a slice has been placed in a viewport of the imaging device 102.

At [C], the imaging device 102 can transmit a signal to the image analysis system 104 representing the captured image data (e.g., the block data and the slice data). The imaging device 102 can send the captured image data as an electronic signal to the image analysis system 104 via the network 108. The signal may include and/or correspond to a pixel representation of the block data and/or the slice data. It will be understood that the signal can include and/or correspond to more, less, or different image data. For example, the signal may correspond to multiple slices of a tissue block and may represent a first slice data and a second slice data. Further, the signal may enable the image analysis system 104 to reconstruct the block data and/or the slice data. In some embodiments, the imaging device 102 can transmit a first signal corresponding to the block data and a second signal corresponding to the slice data. In other embodiments, a first imaging device can transmit a signal corresponding to the block data and a second imaging device can transmit a signal corresponding to the slice data.

At [D], the image analysis system 104 can perform image analysis on the block data and the slice data provided by the imaging device 102. In order to perform the image analysis, the image analysis system 104 may utilize one or more image analysis modules that can perform one or more image processing functions. For example, the image analysis module may include an imaging algorithm, a machine learning model, a convolutional neural network, or any other modules for performing the image processing functions. Based on performing the image processing functions, the image analysis module can determine a likelihood that the block data and the slice data correspond to the same tissue block. For example, an image processing functions may include an edge analysis of the block data and the slice data and based on the edge analysis, determine whether the block data and the slice data correspond to the same tissue block. The image analysis system 104 can obtain a confidence threshold from the user computing device 106, the imaging device 102, or any other device. In some embodiments, the image analysis system 104 can determine the confidence threshold based on a response by the user computing device 106 to a particular recommendation. Further, the confidence threshold may be specific to a user, a group of users, a type of tissue block, a location of the tissue block, or any other factor. The image analysis system 104 can compare the determined confidence threshold with the image analysis performed by the image analysis module. For example, the image analysis system 104 can provide a diagnosis regarding whether the image data is indicative of a disease present in the tissue sample, for example, based on the results of a machine learning algorithm.

At [E], the image analysis system 104 can transmit a signal to the user computing device 106. The image analysis system 104 can send the signal as an electrical signal to the user computing device 106 via the network 108. The signal may include and/or correspond to a representation of the diagnosis. Based on receiving the signal, the user computing device 106 can determine the diagnosis. In some embodiments, the image analysis system 104 may transmit a series of recommendations corresponding to a group of tissues blocks and/or a group of slices. The image analysis system 104 can include, in the recommendation, a recommended action of a user. For example, the recommendation may include a recommendation for the user to review the tissue block and the slice. Further, the recommendation may include a recommendation that the user does not need to review the tissue block and the slice.

### Imaging Prepared Blocks and Prepared Slices

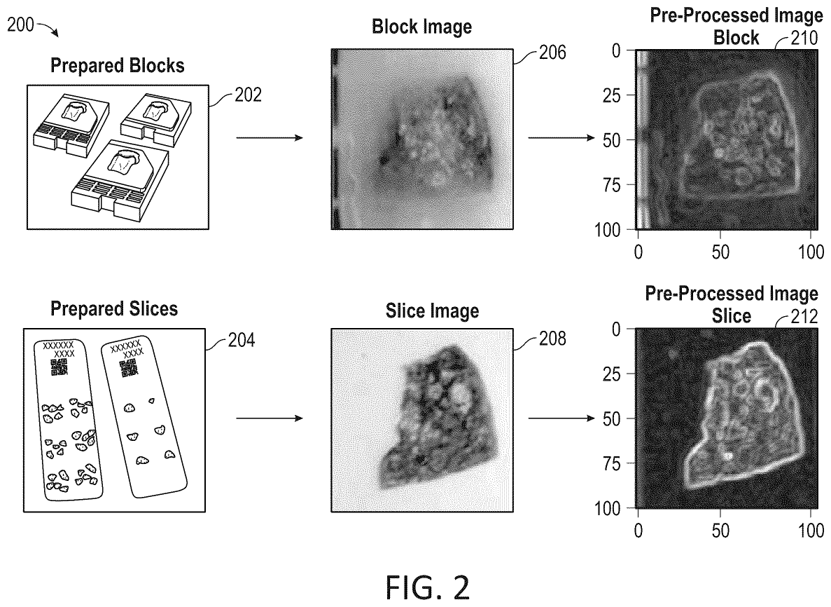

FIG. 2 depicts an example workflow 200 for generating image data from a tissue sample block according to some embodiments. The example workflow 200 illustrates a process for generating prepared blocks and prepared slices from a tissue block and generating pre-processed images based on the prepared blocks and the prepared slices. The example workflow 200 may be implemented by one or more computing devices. For example, the example workflow 200 may be implemented by a microtome, a coverslipper, a stainer, and an imaging device. Each computing device may perform a portion of the example workflow. For example, the microtome may cut the tissue block in order to generate one or more slices of the tissue block. The coverslipper or microtome may be used to create a first slide for the tissue block and/or a second slide for a slice of the tissue block, the stainer may stain each slide, and the imaging device may image each slide.

A tissue block can be obtained from a patient (e.g., a human, an animal, etc.). The tissue block may correspond to a section of tissue from the patient. The tissue block may be surgically removed from the patient for further analysis. For example, the tissue block may be removed in order to determine if the tissue block has certain characteristics (e.g., if the tissue block is cancerous). In order to generate the prepared blocks 202, the tissue block may be prepared using a particular preparation process by a tissue preparer. For example, the tissue block may be preserved and subsequently embedded in a paraffin wax block. Further, the tissue block may be embedded (in a frozen state or a fresh state) in a block. The tissue block may also be embedded using an optimal cutting temperature (“OCT”) compound. The preparation process may include one or more of a paraffin embedding, an OCT-embedding, or any other embedding of the tissue block. In the example of FIG. 2, the tissue block is embedded using paraffin embedding. Further, the tissue block is embedded within a paraffin wax block and mounted on a microscopic slide in order to formulate the prepared block.

The microtome can obtain a slice of the tissue block in order to generate the prepared slices 204. The microtome can use one or more blades to slice the tissue block and generate a slice (e.g., a section) of the tissue block. The microtome can further slice the tissue block to generate a slice with a preferred level of thickness. For example, the slice of the tissue block may be 1 millimeter. The microtome can provide the slice of the tissue block to a coverslipper. The coverslipper can encase the slice of the tissue block in a slide to generate the prepared slices 204. The prepared slices 204 may include the slice mounted in a certain position. Further, in generating the prepared slices 204, a stainer may also stain the slice of the tissue block using any staining protocol. Further, the stainer may stain the slice of the tissue block in order to highlight certain portions of the prepared slices 204 (e.g., an area of interest). In some embodiments, a computing device may include both the coverslipper and the stainer and the slide may be stained as part of the process of generating the slide.

The prepared blocks 202 and the prepared slices 204 may be provided to an imaging device for imaging. In some embodiments, the prepared blocks 202 and the prepared slices 204 may be provided to the same imaging device. In other embodiments, the prepared blocks 202 and the prepared slices 204 are provided to different imaging devices. The imaging device can perform one or more imaging operations on the prepared blocks 202 and the prepared slices 204. In some embodiments, a computing device may include one or more of the tissue preparer, the microtome, the coverslipper, the stainer, and/or the imaging device.

The imaging device can capture an image of the prepared block 202 in order to generate the block image 206. The block image 206 may be a representation of the prepared block 202. For example, the block image 206 may be a representation of the prepared block 202 from one direction (e.g., from above). The representation of the prepared block 202 may correspond to the same direction as the prepared slices 204 and/or the slice of the tissue block. For example, if the tissue block is sliced in a cross-sectional manner in order to generate the slice of the tissue block, the block image 206 may correspond to the same cross-sectional view. In order to generate the block image 206, the prepared block 202 may be placed in a cradle of the imaging device and imaged by the imaging device. Further, the block image 206 may include certain characteristics. For example, the block image 206 may be a color image with a particular resolution level, clarity level, zoom level, or any other image characteristics.

The imaging device can capture an image of the prepared slices 204 in order to generate the slice image 208. The imaging device can capture an image of a particular slice of the prepared slices 204. For example, a slide may include any number of prepared slices and the imaging device may capture an image of a particular slice of the prepared slices. The slice image 208 may be a representation of the prepared slices 204. The slice image 208 may correspond to a view of the slice according to how the slice of the tissue block was generated. For example, if the slice of the tissue block was generated via a cross-sectional cut of the tissue block, the slice image 208 may correspond to the same cross-sectional view. In order to generate the slice image 208, the slide containing the prepared slices 204 may be placed in a cradle of the imaging device (e.g., in a viewer of a microscope) and imaged by the imaging device. Further, the slice image 208 may include certain characteristics. For example, the slice image 208 may be a color image with a particular resolution level, clarity level, zoom level, or any other image characteristics.

The imaging device can process the block image 206 in order to generate a pre-processed image 210 and the slice image 208 in order to generate the pre-processed image 212. The imaging device can perform one or more image operations on the block image 206 and the slice image 208 in order to generate the pre-processed image 210 and the pre-processed image 212. The one or more image operations may include isolating (e.g., focusing on) various features of the pre-processed image 210 and the pre-processed imaged 212. For example, the one or more image operations may include isolating the edges of a slice or a tissue block, isolating areas of interest within a slice or a tissue block, or otherwise modifying (e.g., transforming) the block image 206 and/or the slice image 208. In some embodiments, the imaging device can perform the one or more image operations on one of the block image 206 or the slice image 208. For example, the imaging may perform the one or more image operations on the block image 206. In other embodiments, the imaging device can perform first image operations on the block image 206 and second image operations on the slice image 208. The imaging device may provide the pre-processed image 210 and the pre-processed image 212 to the image analysis system to determine a likelihood that the pre-processed image 210 and the pre-processed image 212 correspond to the same tissue block.

### Slicing a Tissue Block

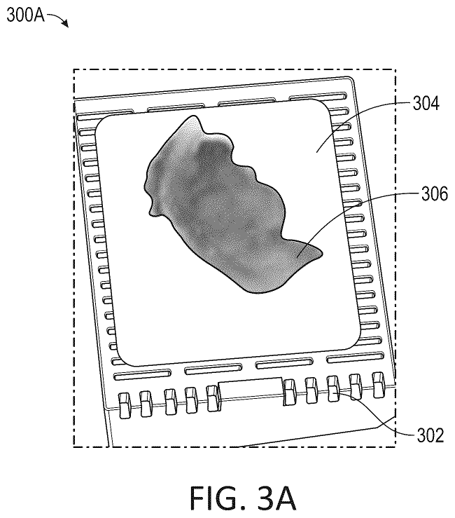

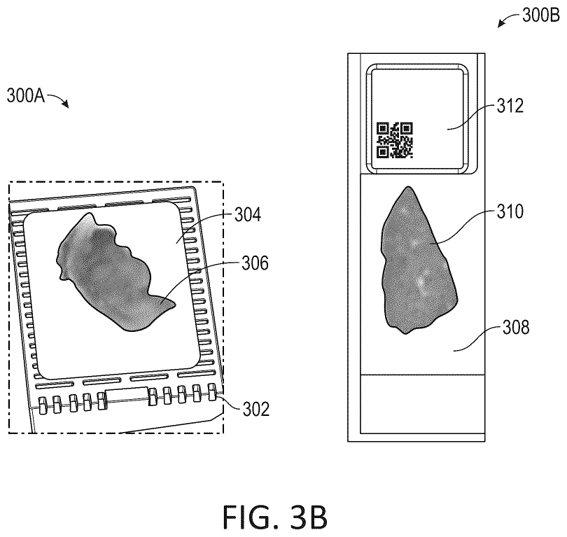

FIG. 3A illustrates an example prepared tissue block 300A according to some embodiments. The prepared tissue block 300A may include a tissue block 306 that is preserved (e.g., chemically preserved, fixed, supported) in a particular manner. In order to generate the prepared tissue block 300A, the tissue block 306 can be placed in a fixing agent (e.g., a liquid fixing agent). For example, the tissue block 306 can be placed in a fixative such as formaldehyde solution. The fixing agent can penetrate the tissue block 306 and preserve the tissue block 306. The tissue block 306 can subsequently be isolated in order to enable further preservation of the tissue block 306. Further, the tissue block 306 can be immersed in one or more solutions (e.g., ethanol solutions) in order to replace water within the tissue block 306 with the one or more solutions. The tissue block 306 can be immersed in one or more intermediate solutions. Further, the tissue block 306 can be immersed in a final solution (e.g., a histological wax). For example, the histological wax may be a purified paraffin wax. After being immersed in a final solution, the tissue block 306 may be formed into a prepared tissue block 300A. For example, the tissue block 306 may be placed into a mould filled with the histological wax. By placing the tissue block in the mould, the tissue block 306 may be moulded (e.g., encased) in the final solution 304. In order to generate the prepared tissue block 300A, the tissue block 306 in the final solution 304 may be placed on a platform 302. Therefore, the prepared tissue block 300A may be generated. It will be understood that the prepared tissue block 300A may be prepared according to any tissue preparation methods.

FIG. 3B illustrates an example prepared tissue block 300A and an example prepared tissue slice 300B according to some embodiments. The prepared tissue block 300A may include the tissue block 306 encased in a final solution 304 and placed on a platform 302. In order to generate the prepared tissue slice 300B, the prepared tissue block 300A may be sliced by a microtome. The microtome may include one or more blades to slice the prepared tissue block 300A. The microtome may take a cross-sectional slice 310 of the prepared tissue block 300A using the one or more blades. The cross-sectional slice 310 of the prepared tissue block 300A may include a slice 310 (e.g., a section) of the tissue block 306 encased in a slice of the final solution 304. In order to preserve the slice 310 of the tissue block 306, the slice 310 of the tissue block 306 may be modified (e.g., washed) to remove the final solution 304 from the slice 310 of the tissue block 306. For example, the final solution 304 may be rinsed and/or isolated from the slice 310 of the tissue block 306. Further, the slice 310 of the tissue block 306 may be stained by a stainer. In some embodiments, the slice 310 of the tissue block 306 may not be stained. The slice 310 of the tissue block 306 may subsequently be encased in a slide 308 by a coverslipper to generate the prepared tissue slice 300B. The prepared tissue slice 300B may include an identifier 312 identifying the tissue block 306 that corresponds to the prepared tissue slice 300B. Not shown in FIG. 3B, the prepared tissue block 300A may also include an identifier that identifies the tissue block 306 that corresponds to the prepared tissue block 300A. As the prepared tissue block 300A and the prepared tissue slice 300B correspond to the same tissue block 306, the identifier of the prepared tissue block 300A and the identifier 312 of the prepared tissue slice 300B may identify the same tissue block 306.

### Imaging Devices

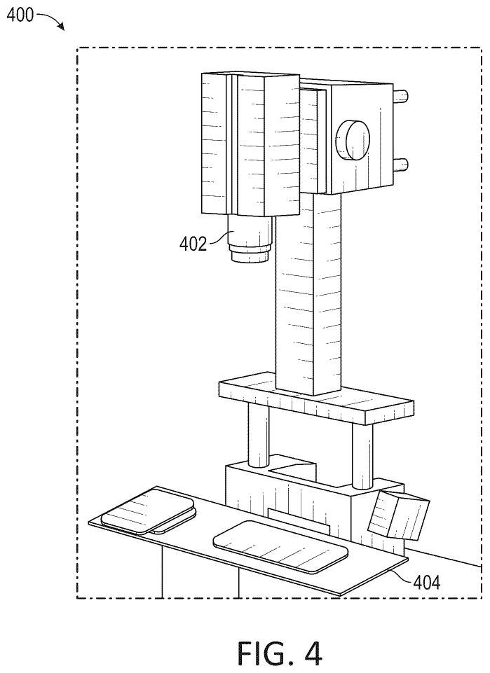

FIG. 4 shows an example imaging device 400, according to one embodiment. The imaging device 400 can include an imaging apparatus 402 (e.g., a lens and an image sensor) and a platform 404. The imaging device 400 can receive a prepared tissue block and/or a prepared tissue slice via the platform 404. Further, the imaging device can use the imaging apparatus 402 to capture image data corresponding to the prepared block and/or the prepared slice. The imaging device 400 can be one or more of a camera, a scanner, a medical imaging device, etc. Further, the imaging device 400 can use imaging technologies such as X-ray radiography, magnetic resonance imaging, ultrasound, endoscopy, elastography, tactile imaging, thermography, medical photography, nuclear medicine functional imaging, positron emission tomography, single-photon emission computed tomography, etc. For example, the imaging device can be a magnetic resonance imaging (“MRI”) scanner, a positron emission tomography (“PET”) scanner, an ultrasound imaging device, an x-ray imaging device, a computerized tomography (“CT”) scanner,

The imaging device 400 may receive one or more of the prepared tissue block and/or the prepared tissue slice and capture corresponding image data. In some embodiments, the imaging device 400 may capture image data corresponding to a plurality of prepared tissue slices and/or a plurality of prepared tissue blocks. The imaging device 400 may further capture, through the lens of the imaging apparatus 402, using the image sensor of the imaging apparatus 402, a representation of a prepared tissue slice and/or a prepared tissue block as placed on the platform. Therefore, the imaging device 400 can capture image data in order for the image analysis system to compare the image data to determine if the image data corresponds to the same tissue block.

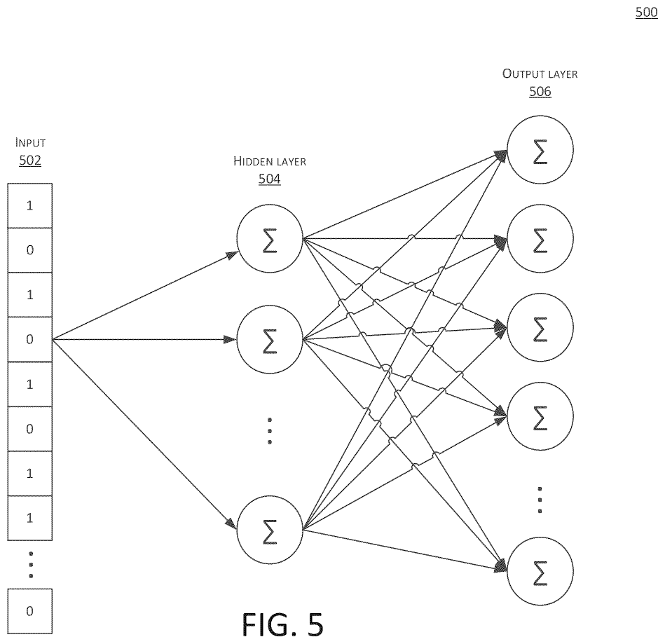

FIG. 5 is an example computing system 500 which can implement any one or more of the imaging device 102, image analysis system 108, and user computing device 110 of the imaging system illustrated in FIG. 1. The computing system 500 may include: one or more computer processors 502, such as physical central processing units (“CPUs”); one or more network interfaces 504, such as a network interface cards (“NICs”); one or more computer readable medium drives 506, such as a high density disk (“HDDs”), solid state drives (“SDDs”), flash drives, and/or other persistent non-transitory computer-readable media; an input/output device interface 508, such as an input/output (“IO”) interface in communication with one or more microphones; and one or more computer readable memories 510, such as random access memory (“RAM”) and/or other volatile non-transitory computer-readable media.

The network interface 504 can provide connectivity to one or more networks or computing systems. The computer processor 502 can receive information and instructions from other computing systems or services via the network interface 504. The network interface 504 can also store data directly to the computer-readable memory 510. The computer processor 502 can communicate to and from the computer-readable memory 510, execute instructions and process data in the computer readable memory 510, etc.

The computer readable memory 510 may include computer program instructions that the computer processor 502 executes in order to implement one or more embodiments. The computer readable memory 510 can store an operating system 512 that provides computer program instructions for use by the computer processor 502 in the general administration and operation of the computing system 500. The computer readable memory 510 can further include computer program instructions and other information for implementing aspects of the present disclosure. For example, in one embodiment, the computer readable memory 510 may include a machine learning model 514 (also referred to as a machine learning algorithm). As another example, the computer-readable memory 510 may include image data 516. In some embodiments, multiple computing systems 500 may communicate with each other via respective network interfaces 504, and can implement multiple sessions each session with a corresponding connection parameter (e.g., each computing system 500 may execute one or more separate instances of the method 700), in parallel (e.g., each computing system 500 may execute a portion of a single instance of the method 700), etc.

### Machine Learning Algorithms

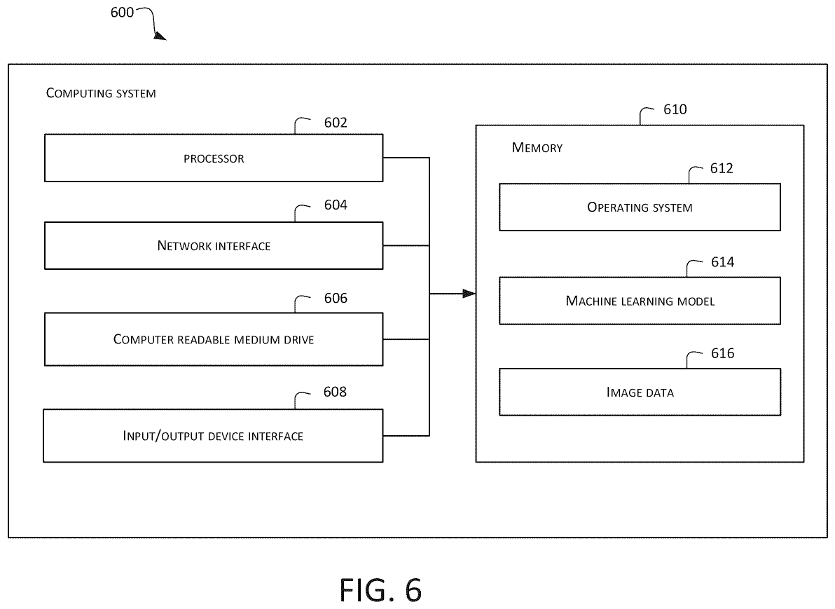

FIG. 6 depicts a schematic diagram of a machine learning algorithm 600, including a multiple layer neural network in accordance with aspects of the present disclosure. The machine learning algorithm 600 can include one or more machine learning algorithms in order to diagnose one or more diseases within image data provided as an input to the machine leaning algorithm 600 by identifying structures or features present in the image data that are consistent with training data used to train the machine learning algorithm 600. Further, the machine learning algorithm 600 may correspond to one or more of a machine learning model, a convolutional neural network, etc.

The machine learning algorithm 600 can include an input layer 602, one or more intermediate layer(s) 604 (also referred to as hidden layer(s)), and an output layer 606. The input layer 602 may be an array of pixel values. For example, the input layer may include a 320×320×3 array of pixel values. Each value of the input layer 602 may correspond to a particular pixel value. Further, the input layer 602 may obtain the pixel values corresponding to the image. Each input of the input layer 602 may be transformed according to one or more calculations.

Further, the values of the input layer 602 may be provided to an intermediate layer 604 of the machine learning algorithm. In some embodiments, the machine learning algorithm 600 may include one or more intermediate layers 604. The intermediate layer 604 can include a plurality of activation nodes that each perform a corresponding function. Further, each of the intermediate layer(s) 604 can perform one or more additional operations on the values of the input layer 602 or the output of a previous one of the intermediate layer(s) 604. For example, the input layer 602 is scaled by one or more weights 603a, 603b, . . . , 603m prior to being provided to a first one of the one or more intermediate layers 604. Each of the intermediate layers 604 includes a plurality of activation nodes 604a, 604b, . . . , 604n. While many of the activation nodes 604a, 604b, . . . are configured to receive input from the input layer 602 or a prior intermediate layer, the intermediate layer 604 may also include one or more activation nodes 604n that do not receive input. Such activation nodes 604n may be generally referred to as bias activation nodes. When an intermediate layer 604 includes one or more bias activation nodes 604n, the number m of weights applied to the inputs of the intermediate layer 604 may not be equal to the number of activation nodes n of the intermediate layer 604. Alternatively, when an intermediate layer 604 does not includes any bias activation nodes 604n, the number m of weights applied to the inputs of the intermediate layer 604 may be equal to the number of activation nodes n of the intermediate layer 604.

By performing the one or more operations, a particular intermediate layer 604 may be configured to produce a particular output. For example, a particular intermediate layer 604 may be configured to identify an edge of a tissue sample and/or a block sample. Further, a particular intermediate layer 604 may be configured to identify an edge of a tissue sample and/or a block sample and another intermediate layer 604 may be configured to identify another feature of the tissue sample and/or a block sample. Therefore, the use of multiple intermediate layers can enable the identification of multiple features of the tissue sample and/or the block sample. By identifying the multiple features, the machine learning algorithm can provide a more accurate identification of a particular image. Further, the combination of the multiple intermediate layers can enable the machine learning algorithm to better diagnose the presence of a disease. The output of the last intermediate layer 604 may be received as input at the output layer 606 after being scaled by weights 605a, 605b, 605m. Although only one output node is illustrated as part of the output layer 606, in other implementations, the output layer 606 may include a plurality of output nodes.

The outputs of the one or more intermediate layers 604 may be provided to an output layer 606 in order to identify (e.g., predict) whether the image data is indicative of a disease present in the tissue sample. In some embodiments, the machine learning algorithm may include a convolution layer and one or more non-linear layers. The convolution layer may be located prior to the non-linear layer(s).

In order to diagnose the tissue sample associated with image data, the machine learning algorithm 600 may be trained to identify a disease. By such training, the trained machine learning algorithm 600 is trained to recognize differences in images and/or similarities in images. Advantageously, the trained machine learning algorithm 600 is able to produce an indication of a likelihood that particular sets of image data are indicative of a disease present in the tissue sample.

Training data associated with tissue sample(s) may be provided to or otherwise accessed by the machine learning algorithm 600 for training. The training data may include image data corresponding to a tissue sample tissue block data that has previously been identified as having a disease. The machine learning algorithm 600 trains using the training data set. The machine learning algorithm 600 may be trained to identify a level of similarity between first image data and the training data. The machine learning algorithm 600 may generate an output that includes a representation (e.g., an alphabetical, numerical, alphanumerical, or symbolical representation) of whether a disease present in a tissue sample corresponding to the first image data.

In some embodiments, training the machine learning algorithm 600 may include training a machine learning model, such as a neural network, to determine relationships between different image data. The resulting trained machine learning model may include a set of weights or other parameters, and different subsets of the weights may correspond to different input vectors. For example, the weights may be encoded representations of the pixels of the images. Further, the image analysis system can provide the trained image analysis module 600 for image processing. In some embodiments, the process may be repeated where a different image analysis module 600 is generated and trained for a different data domain, a different user, etc. For example, a separate image analysis module 600 may be trained for each data domain of a plurality of data domains within which the image analysis system is configured to operate.

Illustratively, the image analysis system may include and implement one or more imaging algorithms. For example, the one or more imaging algorithms may include one or more of an image differencing algorithm, a spatial analysis algorithm, a pattern recognition algorithm, a shape comparison algorithm, a color distribution algorithm, a blob detection algorithm, a template matching algorithm, a SURF feature extraction algorithm, an edge detection algorithm, a keypoint matching algorithm, a histogram comparison algorithm, or a semantic texton forest algorithm. The image differencing algorithm can identify one or more differences between first image data and second image data. The image differencing algorithm can identify differences between the first image data and the second image data by identifying differences between each pixel of each image. The spatial analysis algorithm can identify one or more topological or spatial differences between the first image data and the second image data. The spatial analysis algorithm can identify the topological or spatial differences by identifying differences in the spatial features associated with the first image data and the second image data. The pattern recognition algorithm can identify differences in patterns of the first image data and the training data. The pattern recognition algorithm can identify differences in patterns of the first image data and patterns of the training data. The shape comparison algorithm can analyze one or more shapes of the first image data and one or more shapes of the second image data and determine if the shapes match. The shape comparison algorithm can further identify differences in the shapes.

The color distribution algorithm may identify differences in the distribution of colors over the first image data and the second image data. The blob detection algorithm may identify regions in the first image data that differ in image properties (e.g., brightness, color) from a corresponding region in the training data. The template matching algorithm may identify the parts of first image data that match a template (e.g., training data). The SURF feature extraction algorithm may extract features from the first image data and the training data and compare the features. The features may be extracted based at least in part on particular significance of the features. The edge detection algorithm may identify the boundaries of objects within the first image data and the training data. The boundaries of the objects within the first image data may be compared with the boundaries of the objects within the training data. The keypoint matching algorithm may extract particular keypoints from the first image data and the training data and compare the keypoints to identify differences. The histogram comparison algorithm may identify differences in a color histogram associated with the first image data and a color histogram associated with the training data. The semantic texton forests algorithm may compare semantic representations of the first image data and the training data in order to identify differences. It will be understood that the image analysis system may implement more, less, or different imaging algorithms. Further, the image analysis system may implement any imaging algorithm in order to identify differences between the first image data and the training data.

### Annotation Method and System for Training of Machine Learning Models

As described in connection with FIGS. 1-6, in some implementations, the image analysis system can include an image analysis module (e.g., a machine learning algorithm). The image analysis module may be trained to identify and diagnose one or more diseases by identifying structures or features present in the image data that are consistent with training data used to train the image analysis module. With reference to FIG. 5 (e.g., hardware processor 502 and memory 510 and its components) and FIG. 6 (machine learning (“ML”) algorithm 600 and input layer or data 602), the image analysis system may obtain training data (e.g., input to the ML algorithm 600) that may be fed into the ML model 514 during the training stage. Thus, details about training the ML algorithm 600 (represented by ML model 514) are described herein. Once the ML model 514 is trained with sufficient and correct type of data as described herein, the hardware processor 502 may execute instructions from and in cooperation with the trained ML model 514 to identify and/or stage invasiveness of cancer cells in one or more images obtained from image data 516.

The image analysis system can train ML models (e.g., AI algorithms and processes of ML algorithm 600) for image classification and segmentation. As noted above, Convolutional Neural Networks (CNN) are a type of ML model that may be used for solving this type of problem. In traditional image analysis systems, such a CNN model may be trained by example, whereby humans label and outline features of interest in an image. For example, in traditional image analysis systems, a user may provide a single label for each image (cat, dog, bird, etc.). Where multiple objects may be found within an image, single labels may be unsatisfactory and annotation (e.g., hand drawn outlines) of the image may be required. For example, annotations may be provided via a user interface and a computer having imaging display and annotation capabilities.

Specifically in the field of pathology, objects of interest may correspond to a plurality of different cell types. The objects may vary in size, and may be intermixed in a single sub-image (e.g., a FOV, a portion of an image, a sub-image of an image, etc.). Further, the sub-image may not easily be broken down into smaller images, with one object type per smaller image. In traditional image analysis systems, a labeled drawing on the sub-image may be used to outline each type of object within the sub-image. Due to the complexity found in pathology images, such a manual annotation can be time consuming and may require the subject matter expertise of a trained pathologist.

The image analysis module (e.g., the CNN model) may obtain training data. The process of obtaining the training data may be a time consuming and/or inefficient process. Specifically, the process may be time consuming and/or inefficient due to the annotation process being time consuming, the annotation process requiring input from a particular pathologist, which may be expensive (a limited resource), and/or the annotation process may not be part of a normal workflow of the pathologist (training is required). Further, the process may be time consuming and/or inefficient based on the amount of training data for the image analysis module.

Therefore, in pathology imaging applications, it may be desirable to implement an efficient process for generating and/or collecting training data and training the image analysis module using the training data. One of the many advantages provided by the embodiments of this disclosure is the ability to provide one or more alternatives to hand drawn outlines. With such alternatives, the drawbacks about annotation of images, discussed above, may be improved or avoided. Further, a user may not need the image analysis module to predict the outlines of objects. Therefore, the user may not provide these outlines for training the image analysis module. Instead, the image analysis module may predict a number of objects in a sub-image and a separate system may identify the outlines of the objects.

In one embodiment, a user may designate one or more sub-images on a slide image and record the number of objects present in each sub-image (e.g., the relative percentage of objects present in each sub-image). The number of objects in the sub-image may specify the percentage of objects in the sub-image that are located in the sub-image relative to the image (e.g., the ratio of objects located in a portion of the sub-image to the objects located in the image). For example, the image may include 100 objects and the sub-image may include 25 objects, therefore, the number of objects in the sub-image may be 25%. In some cases, the number of objects in the sub-image may specify a numerical count of the number of objects located in a p sub-image. For example, the image may include 100 objects and the sub-image may include 25 objects, therefore, the number of objects in the sub-image may be 25. In some cases, the number of objects in the sub-image may specify a phrase, a symbolical representation, etc. that represents the number of objects located in a sub-image. For example, the image may include 100 objects and the sub-image may include 25 objects, therefore, the number of objects in the sub-image may be “low” or “−.” It will be understood that the number of objects in the sub-image may include any numerical, alphabetical, alphanumerical, symbolical, etc. representation of the number of objects in the image data.

The user may designate the sub-image via a rectangular, circular, oval, square, triangular, or any regularly or irregularly shaped areas. In some cases, the user may designate the number of objects in a combined image (e.g., an image including multiple sub-images). For example, the user may designate estimated numbers of objects present in the entire image.

The user may designate the number of objects present in each of a plurality of sub-images, to obtain a training data set for training the image analysis module. In some cases, the number of objects present in each of the plurality of sub-images may be estimated using one or more measuring scale indicators (e.g., virtual ruler(s)) in the sub-image from which a user or an area calculation software module (not shown in FIG. 5) may be used to compute a number of objects present in each sub-image relative to the area of the entire image.

Therefore, the image analysis system may train the image analysis module to match the sub-image-level numbers (and a sub-image-level weight as discussed below) using the above described numerical data as input. Further, the image analysis system may train the image analysis module to predict the number of objects in the image at the image-level by collectively aggregating the sub-image-level predictions. One method of aggregating the image-level predictions may include determining a weighted average. The training data set may include a weight for each sub-image. The weight may identify a percentage (e.g., by area) of the image that includes the object(s) of interest. Therefore, a first sub-image that includes less objects than a second sub-image may have a lesser weight than the second sub-image.

The image-level predictions may be aggregated from sub-image-level results into an image-level result by developing and training a second image analysis module. The second image analysis module may obtain the output from the image analysis module as input. The second image analysis module may be trained to identify a weight of the sub-image-level predictions to match an input image-level number.

Therefore, the image analysis module may perform a task of identification of objects in a single step. For example, the task may include a first sub-step to find the objects in an image by outlining the object and a second sub-step to analyze the outlined objects to estimate the number of objects located in the image.

A pathologist may be presented with two tasks when viewing an image: detection and quantification. Detection may be the process of finding objects in the image. Quantitation may be the process of measuring some aspect for each set of objects. An example of a pathology detection problem may be locating the presence of cancer cells in an H&E-stained breast biopsy or excision. The presence of invasive cells may indicate that the cancer is changing location within the body (metastasis) and invading surrounding tissue. If invasive cancer cells are detected, the pathologist may also order a quantitative test for specific protein markers, such as Her2, which may cause a “brown membrane” staining. Her2 is a trans-membranous protein related to EGFR. Like EGFR, Her2 has tyrosine kinase activity. Gene amplification and the corresponding overexpression of Her2 may be found in a variety of tumors, including breast and gastric carcinomas. Without the ML algorithm 600, the pathologist when reviewing the Her2 image may assess the relative percentage of invasive cancer cells according to the intensity of the brown membrane staining. The pathologist may report the percentage of invasive cells that are staining 0+, 1+, 2+, and 3+, with 0+ being no brown staining and 3+ being very intense complete staining of the cell membrane. These percentages may be reported for the entire slide and the pathologist may perform the estimation by considering (in some cases tabulating) the percentages in each sub-image and aggregating these into a single result. Such a process may be inefficient and time consuming.

To train the ML algorithm 600 in the case of invasive cancer cell detection, the image analysis module may train the ML algorithm 600 to indicate the location of sub-images where invasive cancer cells are present. Outlines of the specific cells within the sub-image may not be used to train the ML algorithm 600. For instance, a skilled pathologist may know which cells are invasive when looking at an image and does not need assistance with this task. The same may be true for the Her2 quantification problem being analyzed by the ML algorithm 600. Accordingly, in one embodiment, the ML algorithm 600 may be trained with the number of objects in a sub-image and a weight (e.g., the amount of the sub-image occupied by objects). For example, the ML algorithm 600 may be trained with a number of objects that specifies a percentage of objects in the sub-image as compared to the number of objects in the image. Further, the number of objects may be presented to the ML algorithm in a form of a weight factor. Specifically, the number of objects may identify the number of invasive cancer cells that are staining 0+, 1+, 2+, and 3+ within the sub-image. Based on the training of the ML algorithm 600 with the number of objects and corresponding weights, a user (e.g., a pathologist) can observe the output of the ML algorithm 600 and determine when looking at the sub-image whether the ML algorithm 600 predictions are acceptable or reasonable. After inspecting these results for various sub-images, the user can then assess the image or slide-level prediction and determine whether he/she agrees with the findings of or predications by the ML algorithm 600.

In one embodiment, the user may use color coding of the sub-image results as image overlay to aid in assessing the ML algorithm 600 sub-image-level predictions. For example, the output of the ML algorithm 600 may be displayed with an image overlay that enables a user to color code particular objects (e.g., color code cancerous cells and non-cancerous cells).

Therefore, the image analysis module may receive training data that includes annotations of pathology images as input for the training of the ML algorithm or model 600. The image analysis module may output a prediction of the number of objects in each sub-image (e.g., the presence and relative percentages of different cellular object types). In some embodiments, each annotation may identify a specific sub-image. For example, each annotation may identify a particular FOV (e.g., a rectangular FOV). Further, the annotation may specific coordinates, pixel locations, or other identifying information of the sub-image. For example, the annotation may identify a location of the upper-left and lower-right corner pixel locations, a corner location, a width and height, etc. Each annotation may include the number of objects in each sub-image. For example, each annotation may include a set (P) of N numerical percentages (P: p1, p2, . . . , pN), which correspond to the percentage of each cell type present in the sub-image, for which the image analysis module is to be trained (the set of percentages may sum to 100). Each annotation may further include a single weight (W) (e.g., a percentage weight). The weight may specify a percentage of the sub-image (by area) that includes the objects of interest.

In some embodiments, the set of annotations (e.g., including data identifying the sub-images, a number of objects for each sub-image, and a weight for each sub-image) may be used to train a first ML algorithm or model of the image analysis module. The ML model may obtain as input, the data identifying the sub-images, a number of objects for each sub-image in the set, and a weight for each sub-image in the set. The first ML model may output a prediction, including a number of objects and a weight, for the sub-image for each annotation in the set. The image analysis module may adjust the parameters of the first ML model to minimize error in prediction of the number of objects and a weight across the set of annotations. In some embodiments, the image analysis module may determine a root mean square (RMS) value or prediction error. The image analysis module may adjust the annotation set to increase or decrease representation of individual object classes (e.g., to improve overall prediction accuracy).

In some embodiments, the image analysis system may aggregate the output from the image analysis module for each sub-image into a single image/slide-level result. The image analysis system may determine the single image/slide-level result as the weighted average of the number of objects for each sub-image. The image analysis system may multiply the number of objects for each sub-image by a corresponding weight for the sub-image. The image analysis system may aggregate the weighted number across all sub-images for an entire image. The image analysis system may divide the aggregated and weighted number by a sum of each of the weights.

In some embodiments, a second ML algorithm or model of second image analysis module of the image analysis module may aggregate the output of the first ML model into a single image level result for the entire image. Further, the second ML model may aggregate the number of objects (the predictions) for each sub-image of a plurality of sub-images output by the first ML model. The input to the second ML model may include a number of objects and/or a weight output by the first ML model for each sub-image. The input to the second ML model may also include a set of known image-level numbers of objects (e.g., image-level percentages). The second image analysis module may train the ML model using a set of sub-images with corresponding input data. Further, the second image analysis module may adjust the parameters of the second ML model to minimize error in prediction of the image-level result across the set of sub-images.

In some embodiments, the image analysis system may predict the slide or image-level result by combining the first- and second-ML models into a single ML model. Therefore, the image analysis system may construct (e.g., train and produce) multiple ML algorithms or models or a single ML algorithm or model by one or more of the processes (methods) described above. In some cases, the image analysis system may provide the multiple ML algorithms or models or a single ML algorithm or model to a user (e.g., a consumer such as a pathologist) in computer readable medium (such as on a CD, memory stick, or downloadable from a server via a wired and/or wireless network) to operate on the consumer's computer or server to detect disease such as cancer algorithmically.

One or more of the methods described above may also be performed specifically for Her2 quantification. For example, the numbers of objects may be invasive cell percentages (e.g., 0+, 1+, 2+, and 3+).

Further, one or more of the methods described above may be performed specifically for H&E breast cancer detection or for any type of staining in disease or cancer detection. The numbers of objects may be the percentage of invasive cancer cells in the sub-image. Further, the objects may include in-situ cancer cells, lymphocytes, stroma, normal, abnormal, other types, background, or any combination of cells (including combining one or more cells into a single background class, creating a two-class problem with invasive cancer cells, etc.).

In some cases, the weight may be eliminated by specifying a background (not of interest) percentage. In such a case, the background represents the remainder of the sub-image that does not contain objects of interest. For example, the weight may be 1 for all sub-images and the average may be taken by dividing the weight by the number of sub-images.

### AI Processing Module Functionality

One example of the image analysis module is a convolutional neural network (CNN). The CNN may be designed for tumor finding in a digital pathology histological image, e.g. to classify each image pixel into either a non-tumor class or one of a plurality of tumor classes. While the following example refers to breast cancer tumors, it will be understood that the CNN may identify any class of tumors. The image analysis system may implement the CNN to detect and output a number of invasive and in situ breast cancer cell nuclei automatically. The method is applied to a single input image, such as a whole slide image (WSI), or a set of input images, such as a set of WSIs. Each input image is a digitized, histological image, such as a WSI. In the case of a set of input images, these may be differently stained images of adjacent tissue sections. Staining may include staining with biomarkers as well as staining with conventional contrast-enhancing stains. CNN-based identification of the number of tumors may be faster and/or more efficient than manual outlining and/or CNN-based outlining. Therefore, CNN-based identification of the number of tumors enables an entire image to be processed, rather than only manually annotating selected extracted tiles from the image.

The input image may be a pathology image stained with any one of several conventional stains as discussed in more detail elsewhere in this document. For the CNN, image patches may be extracted of certain pixel dimensions, e.g. 128×128, 256×256, 512×512 or 1024×1024 pixels. It will be understood that the image patches can be of arbitrary size and need not be square, but that the number of pixels in the rows and columns of a patch conform to 2n, where n is a positive integer, since such numbers will generally be more amenable for direct digital processing by a suitable single CPU (central processing unit), GPU (graphics processing unit) or TPU (tensor processing unit), or arrays thereof.

A patch may refer to an image portion taken from a WSI, typically with a square or rectangular shape. In this respect a WSI may contain a billion or more pixels (gigapixel image), so image processing may be applied to patches which are of a manageable size (e.g. ca. 500×500 pixels) for processing by a CNN. The WSI may be processed on the basis of splitting it into patches, analyzing the patches with the CNN, then reassembling the output (image) patches into a probability map of the same size as the WSI. The probability map can then be overlaid, e.g. semi-transparently, on the WSI, or part thereof, so that both the pathology image and the probability map can be viewed together. In that sense the probability map is used as an overlay image on the pathology image. The patches analyzed by the CNN may be of all the same magnification, or may have a mixture of different magnifications, e.g. 5×, 20×, 50× etc. and so correspond to different sized physical areas of the sample tissue. By different magnifications, these may correspond to the physical magnifications with which the WSI was acquired, or effective magnifications obtained from digitally downscaling a higher magnification (i.e. higher resolution) physical image.

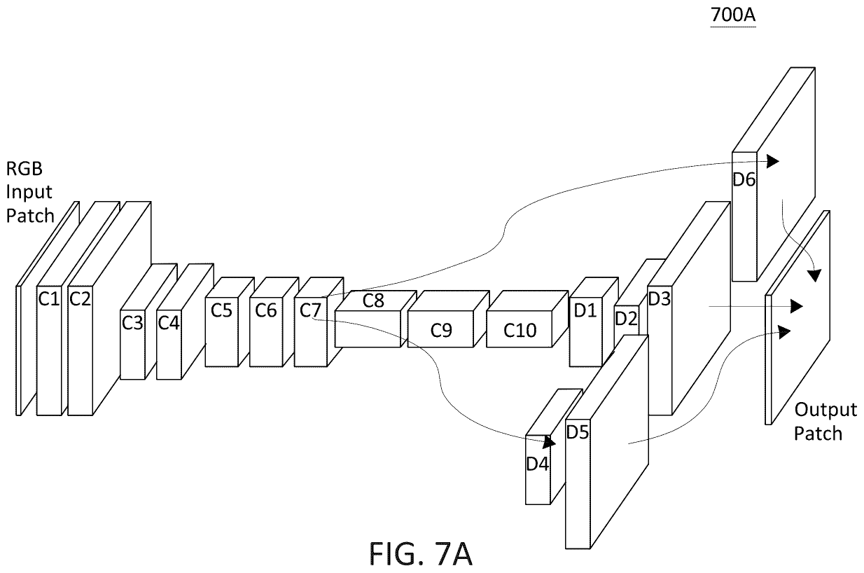

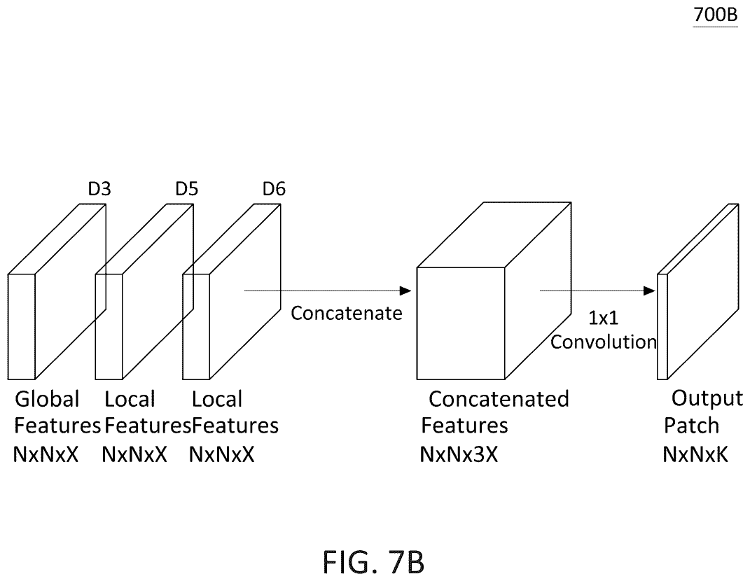

FIG. 7A is a schematic drawing of a neural network architecture. Layers C1, C2 . . . CIO may be convolutional layers. Layers D1, D2, D3, D4, D5 and D6 may be transpose convolution (i.e. deconvolutional) layers. The lines interconnecting certain layers may indicate skip connections between convolutional, C, layers and deconvolutional, D, layers. The skip connections may allow local features from larger dimension, shallower depth layers (where “larger” and “shallow” mean a convolutional layer of lower index) to be combined with the global features from the last (i.e. smallest, deepest) convolutional layer. These skip connections may provide for more accurate outlines. Maxpool layers, each of which is used to reduce the width and height of the patch by a factor of 2, may be present after layers C2, C4 and C7, but are not directly shown in FIG. 7A, although they are shown by implication through the consequential reducing size of the patch. In some implementations of the neural network, the maxpool layers may be replaced with 1×1 convolutions resulting in a fully convolutional network.

The convolutional part of the neural network may have the following layers in sequence: input layer (RGB input image patch); two convolutional layers, C1, C2; a first maxpool layer (not shown); two convolutional layers C3, C4; a second maxpool layer (not shown); three convolutional layers, C5, C6, C7, and a third maxpool layer (not shown). The output from the second and third maxpool layers may be connected directly to deconvolutional layers using skip connections in addition to the normal connections to layers C5 and C8 respectively.

The final convolutional layer, CIO, the output from the second maxpool layer (the layer after layer C4) and the output from the third maxpool layer (the layer after layer C7), may be each connected to separate sequences of “deconvolution layers” which may upscale the outputs to the same size as the input (image) patch. For example, the deconvolution layers can convert the convolutional feature map to a feature map which has the same width and height as the input image patch and a number of channels (e.g., number of feature maps) equal to the number of tissue classes to be detected (e.g., a non-tumorous type and one or more tumorous types). The second maxpool layer may be directly linked to the layer D6 based on only one stage of deconvolution being needed. For the third maxpool layer, two stages of deconvolution may be needed, via intermediate deconvolution layer D4, to reach layer D5. For the deepest convolutional layer CIO, three stages of deconvolution may be needed, via D1 and D2 to layer D3. Therefore, the result may be three arrays D3, D5, D6 of equal size to the input patch.

In some cases, the skip connections may be omitted and layers D4, D5 and D6 may not be present and the output patch may be computed solely from layer D3.

FIG. 7B depicts steps in the neural network architecture of FIG. 7A being carried out. Specifically, global feature map layer D3 and local feature map layers D5, D6 may be combined to generate a feature map that predicts an individual class for each pixel of the input image patch. FIG. 7B illustrates the final three transpose convolution layers D3, D5, D6 that are processed to the tumor class output patch.

To predict the class of individual pixels, the CNN may include convolutional layers with a series of transpose convolutional layers. Therefore, the fully connected layers may be removed from this architecture. Each transpose layer may double the width and height of the feature maps while at the same time halving the number of channels. In this manner, the feature maps may be upscaled back to the size of the input patch. In addition, to improve the prediction, skip connections may be utilized. The skip connections may use shallower features to improve the coarse predictions made by upscaling from the final convolutional layer CIO. The local features from the skip connections contained in layers D5 and D6 of FIG. 7A may be concatenated with the features generated by upscaling the global features contained in layer D3 of FIG. 7A from the final convolutional layer. The global and local feature layers D3, D5 and D6 may then be concatenated into a combined layer as shown in FIG. 7B.

From the concatenated layer of FIG. 7B (or alternatively directly from the final deconvolutional layer D3 in the case that skip connections are not used), the number of channels may be reduced to match the number of classes by a 1×1 convolution of the combined layer. A softmax operation on this classification layer may then convert the values in the combined layer into probabilities. The output patch layer may have a size N*N*K, where N is the width and height in pixels of the input patches and K is the number of classes that are being detected. Therefore, for any pixel P in the image patch there may be an output vector V of size K. A unique class can then be assigned to each pixel P by the location of the maximum value in its corresponding vector V.

The CNN may label each pixel as non-cancerous or belonging to one or more of several different cancer (tumor) types. The cancer types may include breast cancer, cancer of the bladder, colon cancer, rectum cancer, kidney cancer, blood cancer (leukemia), endometrium cancer, lung cancer, liver cancer, skin cancer, pancreas cancer, prostate cancer, brain cancer, spine caner, thyroid cancer, or any other type of cancer. Further, the CNN may label each pixel as belonging to a certain cell type.

The CNN may operate on input images having certain fixed pixel dimensions. Therefore, as a preprocessing step, both for training and prediction, patches may be extracted from the WSI which have the desired pixel dimensions (e.g., N*N*h pixels). For example, N=3 in the case that each physical location has three pixels associated with three primary color (e.g., red, green blue). Further, the WSI may be a color image acquired by a conventional visible light microscope. H may be 3 times the number of composited WSIs in the case the two or more color WSIs are combined. Moreover H may have a value of one in the case of a single monochrome WSI. To make training faster the input patches may be centered and normalized at this stage.

The entire WSI, or at least the entire area of the WSI which contains tissue, may be pre-processed so the patches may be tiles that cover at least the entire tissue area of the WSI. The tiles may be abutting without overlap, or have overlapping edge margin regions of for example 1, 2, 3, 4, 5, 6, 7, 8, 9 or 10 pixels wide so that the output patches of the CNN can be stitched together taking account of any discrepancies. In some cases, the patches may be a random sample of patches over the WSI which may be of the same or different magnification, as provided by a separate system, a user (e.g., a pathologist), etc.

The CNN may use very small 3×3 kernels in all convolutional filters. Max pooling may be performed with a small 2×2 window and stride of 2. The CNN may include convolution layers with a sequence of “deconvolutions” (more accurately transpose convolutions) to generate segmentation masks.

Each deconvolutional layer may enlarge the input feature map (e.g., by a factor of two) in the width and height dimensions. This may counteract the shrinking effect of the maxpool layers and result in class feature maps of the same size as the input images. The output from each convolution and deconvolutional layer may be transformed by a non-linear activation layer. The non-linear activation layers may use the rectifier function ReLU (x)=max (0, x)ReLU(x)=max(0, x). Different activation functions may be used, such as ReLU, leaky ReLU, eLU, etc. as desired.

The CNN can be applied without modification to any desired number of tissue classes. For example, the CNN may identify further breast pathologies including invasive lobular carcinoma or invasive ductal carcinoma (e.g., the single invasive tumor class of the previous example can be replaced with multiple invasive tumor classes).

A softmax regression layer (i.e. multinomial logistic regression layer) may be applied to each of the channel patches to convert the values in the feature map to probabilities.

After this final transformation by the softmax regression, a value at location (x, y) in a channel C in the final feature map may contain the probability, P(x, y), that the pixel at location (x, y) in the input image patch belongs to the tumor type detected by channel C.

The number of convolution and deconvolution layers can be increased or decreased

The neural network may be trained using mini-batch gradient descent. The learning rate may be decreased from an initial rate of 0.1 using exponential decay. Training the network may be done on a GPU, CPU, or a FPGA using any one of several available deep learning frameworks.

The neural network may output probability maps of size N×N×K, where N is the width and height in pixels of the input patches and K is the number of classes that are being detected. These output patches may be stitched back together into a probability map of size W×H×K, where W and H are the width and height of the original WSI before being split into patches. The probability maps can then be collapsed to a W×H label image by recording the class index with maximum probability at each location (x, y) in the label image.

The neural network may assign a pixel to one or more classes (e.g., tissue classes, non-tissue classes, etc.) For example, the neural network may assign the pixel to one of three classes: non-tumor, invasive tumor and in situ tumor. Further, based on assigning a pixel to one or more classes, the neural network may identify a number of objects in the input image. The output image

When multiple tumor classes are used, the output image can be post-processed into a simpler binary classification of non-tumor and tumor (e.g., the multiple tumor classes may be combined). The binary classification may be used as an option when creating images from the base data, while the multi-class tumor classification is retained in the saved data.

While the above description of a particular implementation discusses a specific approach using a CNN, it will be understood that the approach can be implemented in a wide variety of different types of convolutional neural networks. In general, any neural network that uses convolution to detect increasingly complex features and subsequently uses transpose convolutions (“deconvolutions”) to upscale the feature maps back to the width and height of the input image may be suitable.

### Training & Prediction

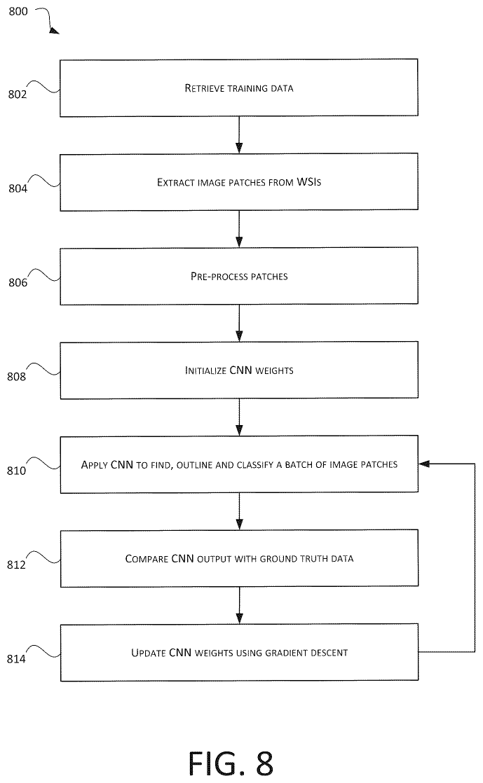

FIG. 8 is a flow diagram showing the steps involved in training the CNN. FIG. 8 shows a method 800 executed by an image analysis module, according to some examples of the disclosed technologies. The image analysis module may be similar, for example, to the image analysis module described above. It will be understood that the method 800 may be performed by different devices (e.g., a computing device). The process 800 may begin automatically upon receiving training data.

In block 802, the image analysis module retrieves training data (e.g., from the image analysis module) containing WSIs for processing which have been annotated by a user to specify a number of objects (e.g., tumors) in the WSIs. The annotations may represent the ground truth data. The training data may include annotations for one or more image patches (e.g., sub-images) of the WSIs. For example, the training day may include annotations for each image patch of a WSI (each image patch corresponding to a portion of the WSI). The image patches together may form the WSI when placed together. In some cases, the image patches may overlap. For example, one or more image patches may include a same portion of the WSI.

The number of objects in the corresponding WSI may identify a percentage of the number of the first plurality of objects in an image patch of the WSI as compared to a number of a plurality of objects in the WSI. For example, the number of objects may identify that the image patch includes 25% of the objects in the WSI. Further, the number of objects may identify a percentage of a particular type of object (e.g., a cancerous cell, a particular type of cancerous cell, etc.) in the image patch as compared to a number of the particular type of object in the WSI. In some cases, the number of objects may identify a numerical count of the number of objects (e.g., 10 objects, 15 objects, etc.). In other cases, the number of objects may specify an alphabetical, symbolical, numerical, alphanumerical, etc. quantification of the number of objects.

The training data may further include one or more weights. For example, the training data may include a weight for each image patch. The weight may identify a portion of the image patch that includes an object or one or more types of objects. For example, the weight for an image patch may specify that 25% of the image patch is occupied by cancerous cells. Further, the weight for an image patch may specify that 25% of the image patch is occupied by stroma.

The training data may further include a designation of the image patches of the WSIs. For example, the training data may identify how the WSIs are to be separated into image patches. The training data may indicate an outline of each image patch. Specifically, the training data may specify coordinates (e.g., pixel coordinates, pixel locations, etc.) of the outline of each image patch. For example, the training data may specify an upper-left corner pixel location and a lower-right corner pixel location that define an outline of the image patch. In some cases, the training data may specify a width, a height, or any other measurements to identify an image patch. Further, the training data may identify a weight and/or a number of objects in the corresponding image patch.

In block 804, the image analysis module extracts image patches from the WSIs (e.g., the image analysis module may break the WSIs down into image patches). The image analysis module may extract the image patches for input as the input image patches to the CNN. The image analysis module may extract the image patches based on the training data.

In block 806, the image analysis module pre-processes the image patches. Alternatively, or in addition, the WSIs could be pre-processed.

In block 808, the image analysis module initializes (e.g., sets) initial values for the CNN weights (e.g., the weights between layers).

In block 810, the image analysis module applies the CNN to find, outline, and classify a batch of image patches based on a batch of input image patches that is input into the CNN. The CNN may find, outline, and classify the batch of image patches on a pixel-by-pixel basis. Further, the image analysis module may analyze the outlined and classified patches to determine a number of objects in each image patch. In some cases, the image analysis may count the number of objects (e.g., the number of a particular type of object) in each patch based on the outlined and classified patches.

The image analysis module may further analyze the outlined and classified patches to determine one or more weights. For example, the image analysis module may determine a weight for each image patch. The weight may identify a portion of the image patch that includes an object or one or more types of objects. The image analysis module may generate CNN output image patches that identify the number of objects in each image patch, a weight for each image patch, and an identification of the image patch.

In block 812, the image analysis module compares CNN output image patches with the ground truth data. This may be done on a per-patch basis. Alternatively, if patches have been extracted that cover the entire WSI, then this may be done at the WSI level, or in sub-areas of the WSI made up of a contiguous batch of patches, e.g. one quadrant of the WSI. In such variants, the output image patches can be reassembled into a probability map for the entire WSI, or contiguous portion thereof, and the probability map can be compared with the ground truth data both by the computer and also by a user visually if the probability map is presented on the display as a semi-transparent overlay to the WSI, for example.

In block 814, the image analysis module updates the CNN weights (e.g., using a gradient descent approach). For example, the image analysis module may learn and update based comparing the CNN output image patches with the ground truth data. Therefore, learning may be fed back into repeated processing of the training data as indicated in FIG. 8 by the return loop in the process flow, so that the CNN weights can be optimized. Based on training of the CNN, the CNN can be applied to WSIs independently of any ground truth data.

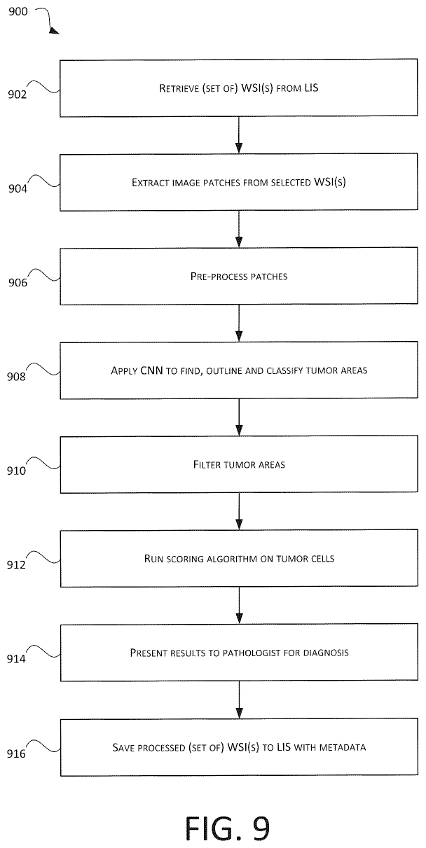

FIG. 9 is a flow diagram showing the steps involved in prediction using the CNN. FIG. 9 shows a method 900 executed by an image analysis module, according to some examples of the disclosed technologies. The image analysis module may be similar, for example, to the image analysis module described above. It will be understood that the method 900 may be performed by different devices (e.g., a computing device). The process 900 may begin automatically upon receiving image data.

In block 902, the image analysis module retrieves one or more (e.g., a set of) WSIs for processing (e.g., from a laboratory information system (LIS) or other histological data repository). The WSIs may be pre-processed.

In block 904, the image analysis module extracts the image patches from the selected WSIs. The patches may cover the entire WSI or may be a random or non-random selection.

In block 906, the image analysis module pre-processes the image patches.

In block 908, the image analysis module applies the CNN to find, outline, and classify tumor areas. Each of a batch of input image patches may be input into the CNN and processed to find, outline, and classify the patches on a pixel-by-pixel basis. The output patches can then be reassembled as a probability map for the WSI from which the input image patches were extracted. The probability map can be compared with the WSI both by the computer apparatus in digital processing and also by a user visually, if the probability map is presented on the display as a semi-transparent overlay on the WSI or alongside the WSI, for example.

In block 910, the image analysis module filters the tumor areas excluding tumors that are likely to be false positives (e.g., areas that are too small or areas that may be edge artifacts).

In block 912, the image analysis module runs a scoring algorithm (e.g., on tumor cells). The scoring may be cell specific and the score may be aggregated for each tumor, and/or further aggregated for the WSI (or sub-area of the WSI).

In block 914, the image analysis module presents the results to a pathologist or other user (e.g., a relevantly skilled clinician) for diagnosis (e.g., by display of the annotated WSI on a suitable high-resolution monitor).

In block 916, the image analysis module saves the processed (set of) WSIs to the LIS with metadata. Therefore, the image analysis module may save the results of the CNN (e.g., the probability map data and optionally also metadata relating to the CNN parameters together with any additional diagnostic information added by the pathologist) in a way that is linked to the patient data file containing the WSI, or set of WSIs, that have been processed by the CNN. The patient data file in the LIS or other histological data repository may be supplemented with the CNN results.

### CNN Computing Platform

The proposed image processing may be carried out on a variety of computing architectures, in particular ones that are optimized for neural networks, which may be based on CPUs, GPUs, TPUs, FPGAs and/or ASICs. In some embodiments, the neural network may be implemented using Google's Tensorflow software library running on Nvidia GPUs from Nvidia Corporation, Santa Clara, California, such as the Tesla K80 GPU. In other embodiments, the neural network can run on generic CPUs.

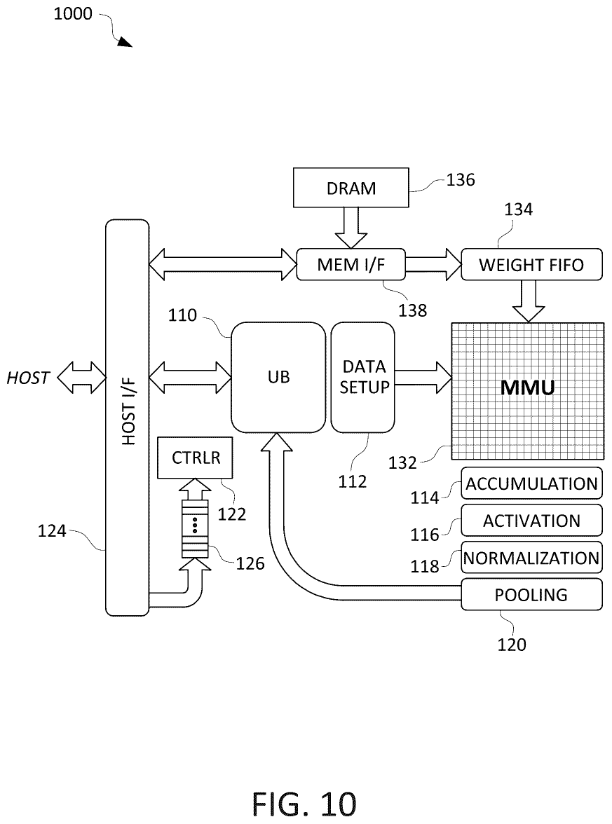

FIG. 10 shows an example TPU 1000. The TPU 1000 has a systolic matrix multiplication unit (MMU) 132 which may contain 256×256 MACs that can perform 8-bit multiply-and-adds on signed or unsigned integers. The weights for the MMU may be supplied through a weight FIFO buffer 134 that may in turn read the weights from a memory 136, in the form of an off-chip 8 GB DRAM, via a suitable memory interface 138. A unified buffer (UB) 110 may be provided to store the intermediate results. The MMU 132 may be connected to receive inputs from the weight FIFO interface 134 and the UB 110 (via a systolic data setup unit 112) and outputs the 16-bit products of the MMU processing to an accumulator unit 114. An activation unit 116 may perform nonlinear functions on the data held in the accumulator unit 114. After further processing by a normalizing unit 118 and a pooling unit 120, the intermediate results may be sent to the UB 110 for resupply to the MMU 132 via the data setup unit 112. The pooling unit 120 may perform maximum pooling (i.e. maxpooling) or average pooling as desired. A programmable DMA controller 122 may transfer data to or from the TPU's host computer and the UB 110. The TPU instructions may be sent from the host computer to the controller 122 via a host interface 124 and an instruction buffer 126.

It will be understood that the computing power used for running the neural network, whether it be based on CPUs, GPUs or TPUs, may be hosted locally in a clinical network, e.g. the one described below, or remotely in a data center.

### Network & Computing & Scanning Environment

The proposed computer-automated method operates in the context of a laboratory information system (LIS) which in turn is typically part of a larger clinical network environment, such as a hospital information system (HIS) or picture archiving and communication system (PACS). In the LIS, the WSIs will be retained in a database, typically a patient information database containing the electronic medical records of individual patients. The WSIs will be taken from stained tissue samples mounted on slides, the slides bearing printed barcode labels by which the WSIs are tagged with suitable metadata, since the microscopes acquiring the WSIs are equipped with barcode readers. From a hardware perspective, the LIS will be a conventional computer network, such as a local area network (LAN) with wired and wireless connections as desired.

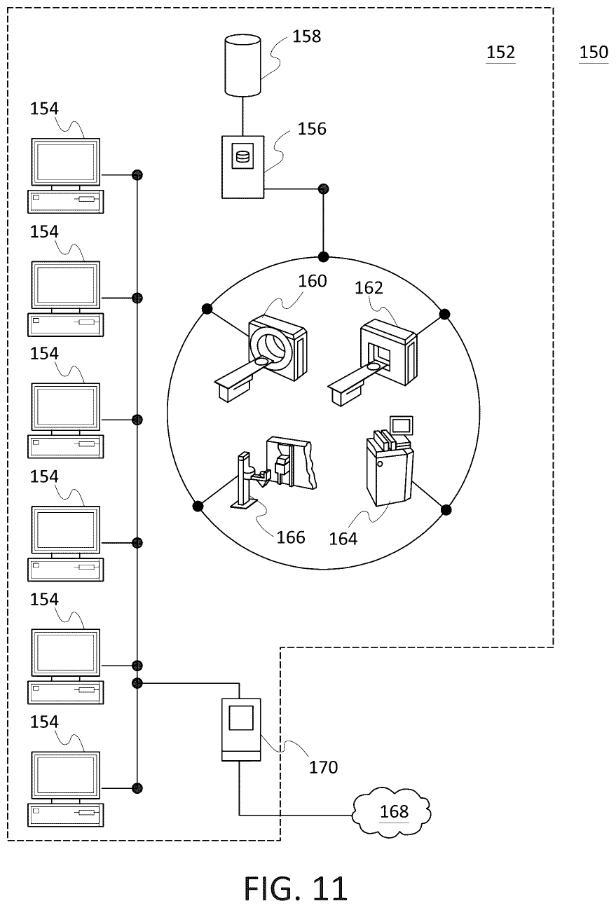

FIG. 11 shows an example computer network which can be used in conjunction with embodiments of the invention. The network 150 comprises a LAN in a hospital 152. The hospital 152 is equipped with a number of workstations 154 which each have access, via the local area network, to a hospital computer server 156 having an associated storage device 158. A LIS, HIS or PACS archive is stored on the storage device 158 so that data in the archive can be accessed from any of the workstations 154. One or more of the workstations 154 has access to a graphics card and to software for computer-implementation of methods of generating images as described hereinbefore. The software may be stored locally at each workstation 154 or may be stored remotely and downloaded over the network 150 to a workstation 154 when needed. In other example, methods embodying the invention may be executed on the computer server with the workstations 154 operating as terminals. For example, the workstations may be configured to receive user input defining a desired histological image data set and to display resulting images while CNN analysis is performed elsewhere in the system. Also, a number of histological and other medical imaging devices 160, 162, 164, 166 are connected to the hospital computer server 156. Image data collected with the devices 160, 162, 164, 166 can be stored directly into the LIS, HIS or PACS archive on the storage device 156. Thus, histological images can be viewed and processed immediately after the corresponding histological image data are recorded. The local area network is connected to the Internet 168 by a hospital Internet server 170, which allows remote access to the LIS, HIS or PACS archive. This is of use for remote accessing of the data and for transferring data between hospitals, for example, if a patient is moved, or to allow external research to be undertaken.

### Example Annotations

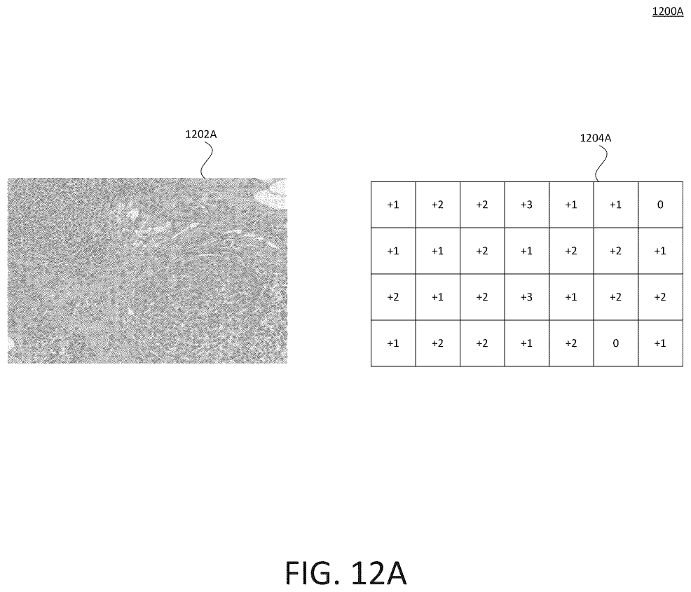

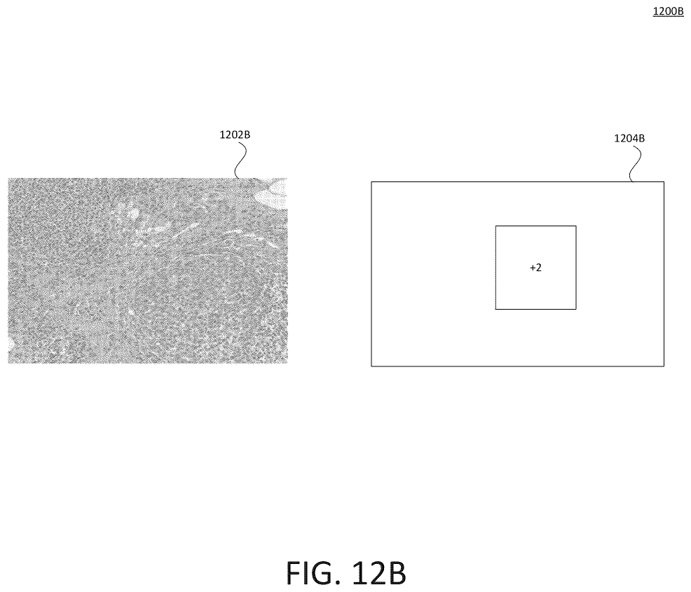

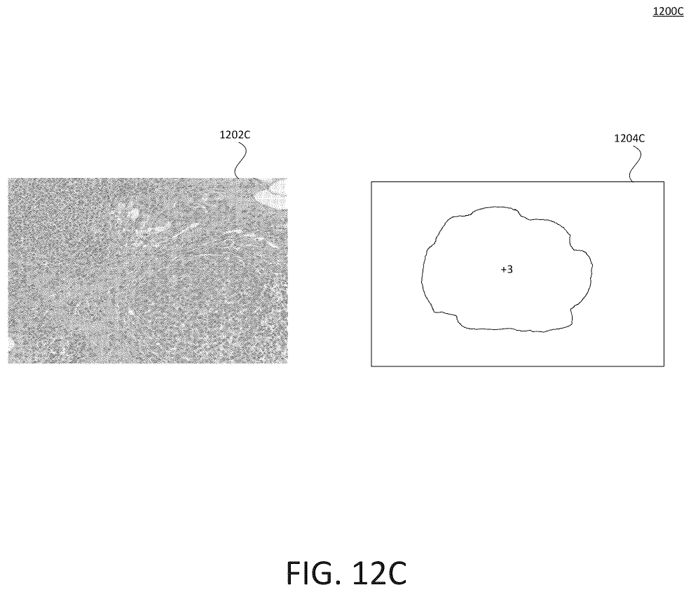

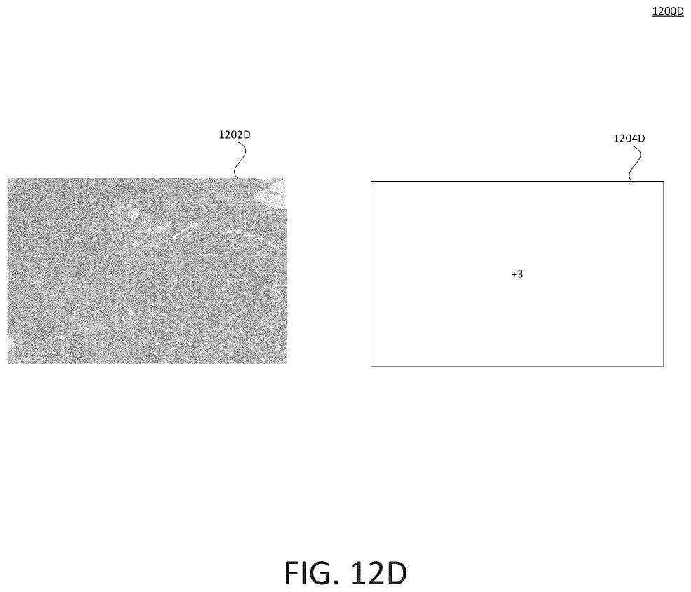

FIGS. 12A, 12B, 12C, and 12D each illustrate an example image and an example numerical representation of objects in the example image. Each of the example images may include a slide image. For example, each of the example images may include a slide image corresponding to a slide of a tissue block. As discussed above, a slice of a tissue block may be encased in a slide to generate a prepared tissue slice. Further, the prepared tissue slice may be imaged (e.g., using an imaging system) and a slide image may be generated.

FIG. 12A depicts a slide image 1202A and a corresponding annotation 1204A. The slide image 1202A may be a color or non-color image of a prepared tissue slice (or any other slide). The slide image 1202A may include one or more objects (e.g., cells). For example, the slide image 1202A may include one or more cancerous cells, one or more non-cancerous cells, etc. Specifically, the slide image 1202A may include in-situ cancer cells, lymphocytes, stroma, normal, abnormal, other types, background, or any combination of cells (including combining one or more cells into a single background class, creating a two-class problem with invasive cancer cells, etc.). The slide image 1202A may be uploaded to the image analysis system for processing (e.g., identification of a number of objects in the slide image 1202A). In some embodiments, the slide image 1202A may be uploaded (e.g., by a user) to the image analysis system as a portion of a training data set for training an image analysis module. In other embodiments, the slide image 1202A may be output (e.g., by user) by the image analysis module to enable a user to identify a number of objects in the slide image 1202A based on the output of the image analysis module.

The annotation 1204A may identify annotated data for the slide image 1202A. The annotation 1204A may identify one or more numbers of objects for one or more patches of the slide image 1202A. The number of objects in a particular patch may identify the number of objects in the patch as compared to the number of objects in the overall image. Further, the number of objects may correspond to a particular type of object. For example, the number of objects may be a number of cancerous cells, a number of background cells, a number of breast cancer cells, etc. In some embodiments, each patch may be associated with multiple numbers of objects. For example, a patch may be associated with a first number of objects identifying a number of background cells, a second number of objects identifying a number of breast cancer cells, and a third number of objects identifying a number of stroma. In some embodiments, an object may be identified as one particular type of object. In other embodiments, an object may be identified as multiple types of objects.

The annotation 1204A may include numerical, alphabetical, alphanumerical, symbolical, or any other data identifying one or more numbers of objects for one or more patches of the slide image 1202. The image analysis system may identify boundaries between different numbers of objects (e.g., based on analysis on the number of objects in each of the patches). For example, the image analysis system may analyze the number of objects in each patch and determine that 50% of the patches are associated with less than 5 objects, 60% of the patches are associated with less than 10 objects, and 90% of the patches are associated with less than 20 objects. Further, the image analysis system may determine, based on the analysis, a patch with 0 objects has a rating of 0, a patch with between 1 and 4 objects has a rating +1, a patch with between 5 and 9 objects has a rating of +2, a patch with between 10 and 19 objects has a rating of +3, and a patch with over 20 objects has a rating of +4. Based on the ratings, the image analysis system may determine how a slide image 1202A is annotated and how to generate annotation 1204A for display.

In some embodiments, the annotation 1204A may further include data identifying the patch for each of the patches (not shown above). For example, the data identifying the patch may specify an outline, a boundary, etc. of the patch. Further, the data identifying the patch may include one or more measurements of the patch. For example, the data identifying the patch may include pixel coordinates (e.g., corner coordinates), a height, a width, a center, a radius, a circumference, etc. of the patch.

In some embodiments, the annotation 1204A may further include a weight for each of the patches (not shown above). The weight may identify a portion of the patch that includes the objects. For example, the weight may identify a percentage of the total area occupied by the patch that is occupied by objects. Further, the weight may be equal to the area within the patch occupied by objects divided by the total area within the patch.

In the example of FIG. 12A, the slide image 1202A is segmented into a plurality of patches (e.g., by the image analysis system, by a user, etc.). The annotated data includes a rating for each patch between 0, +1, +2, and +3. The rating may identify the number of objects within a respective patch. For example, a patch with a rating of 0 may not include any objects, a patch with a rating of 1 may include a low number of objects (e.g., 10 objects, 10% of the overall objects, etc.), a patch with a rating of +2 may include a medium number of objects (e.g., 20 objects, 20% of the overall objects, etc.), and a patch with a rating of +3 may include a high number of objects (e.g., 50 objects, 50% of the overall objects, etc.). Therefore, the annotation 1204A may specify a rating for each of the patches of the slide image 1202A.

In some embodiments, the annotation 1204A may be provided by a user (e.g., via a display of a user interface of a user computing device). For example, the image analysis system may cause display of a customized user interface that displays a representation of the slide image 1202A. Further, the customized user interface may enable the selection of a particular patch and an identification of a number of objects in a particular patch within the slide image 1202A. For example, the customized user interface may enable a user to interact with and/or define a particular patch (e.g., by drawing or outlining the patch on or via the slide image 1202A) and identify and/or define the number of objects in the particular patch and/or a weight for the particular patch.

FIG. 12B depicts a slide image 1202B and a corresponding annotation 1204B as discussed above. In the example of FIG. 12B, a patch is defined in the slide image 1202B (e.g., by the image analysis system, by a user, etc.). The annotated data includes a rating for the patch between 0, +1, +2, and +3. As discussed above, the rating may identify the number of objects within a respective patch. In the example of FIG. 12B, the annotation 1204B identifies that the patch has a rating of +2. Therefore, the annotation 1204B may specify a rating for each of the patches of the slide image 1202B.

In some embodiments, the annotation 1204B may be provided by a user (e.g., via a display of a user interface of a user computing device). For example, the image analysis system may cause display of a customized user interface that displays a representation of the slide image 1202B. Further, the customized user interface may enable the selection and/or definition of a particular patch and an identification of a number of objects in a particular patch within the slide image 1202B. For example, the customized user interface may enable a user to interact with to select the patch and/or define the patch (e.g., by clicking and dragging, drawing a rectangular shape, etc.) and identify and/or define the number of objects in the particular patch and/or a weight for the particular patch.

FIG. 12C depicts a slide image 1202C and a corresponding annotation 1204C as discussed above. In the example of FIG. 12C, a patch is defined in the slide image 1202C (e.g., by the image analysis system, by a user, etc.). The annotated data includes a rating for the patch between 0, +1, +2, and +3. As discussed above, the rating may identify the number of objects within a respective patch. In the example of FIG. 12C, the annotation 1204C identifies that the patch has a rating of +3. Therefore, the annotation 1204C may specify a rating for each of the patches of the slide image 1202C.

In some embodiments, the annotation 1204C may be provided by a user (e.g., via a display of a user interface of a user computing device). For example, the image analysis system may cause display of a customized user interface that displays a representation of the slide image 1202C. Further, the customized user interface may enable the custom selection and/or definition of a particular patch and an identification of a number of objects in a particular patch within the slide image 1202C. For example, the customized user interface may enable a user to interact with to select the patch and/or define the patch (e.g., via a custom drawing of the patch (free hand) and identify and/or define the number of objects in the particular patch and/or a weight for the particular patch. In some embodiments, where the user is providing annotations for training the image analysis module, the image analysis may store data identifying the patch (e.g., based on an identified definition) and the number of objects). In other embodiments, where the user interface is display the output of the image analysis module, the customized user interface may enable a user to approve or disapprove of the annotations 1204C. Based on the user's approval of the annotation 1204C (e.g., routed by the user computing device to the image analysis module), the image analysis module may generate additional annotations. Based on the user's disapproval of the annotations 1204C (e.g., routed by the user computing device to the image analysis module), the image analysis system may train the image analysis module using additional training data (e.g., provided by the user computing device).

FIG. 12D depicts a slide image 1202D and a corresponding annotation 1204D as discussed above. In the example of FIG. 12D, a patch is not defined in the slide image 1202D and the annotation 1204D may include annotation data for the entire slide image 1202D. The annotated data includes a rating for the slide image 1202D between 0, +1, +2, and +3. As discussed above, the rating may identify the number of objects within slide image 1202D. In the example of FIG. 12D, the annotation 1204D identifies that slide image 1202D has a rating of +3. Therefore, the annotation 1204D may specify a rating for the slide image 1202D.

In some embodiments, the annotation 1204D may be provided by a user (e.g., via a display of a user interface of a user computing device). For example, the image analysis system may cause display of a customized user interface that displays a representation of the slide image 1202D. Further, the customized user interface may enable the selection and/or definition of slide image 1202D and an identification of a number of objects in the slide image 1202D. For example, the customized user interface may enable a user to interact with and/or define t slide image 1202D and identify and/or define the number of objects in the slide image 1202D and/or a weight for the slide image 1202D.

### Prediction of a Number of Objects

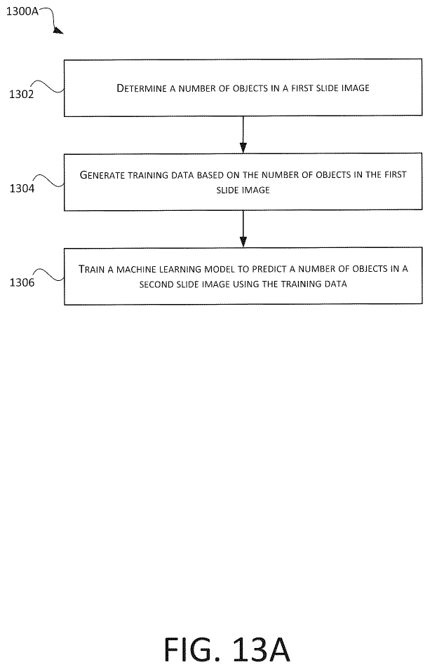

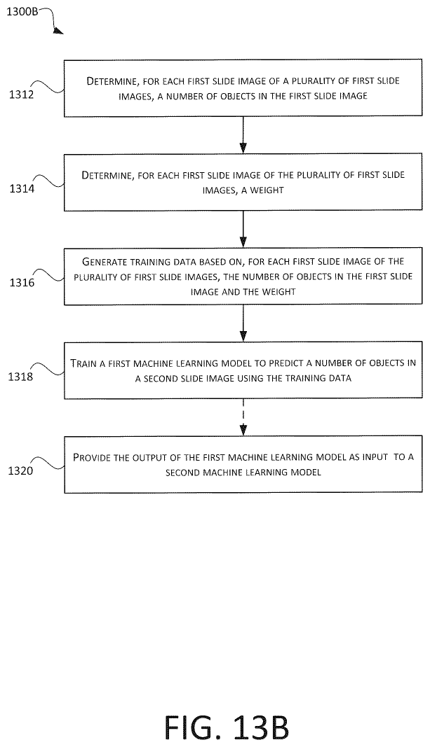

FIG. 13A is a flow diagram showing the steps involved in training the image analysis module. FIG. 13A shows a method 1300A executed by an image analysis system, according to some examples of the disclosed technologies. The image analysis system may be similar, for example, to the image analysis system described above. It will be understood that the method 1300A may be performed by different devices (e.g., a computing device). The method 1300A may begin automatically upon receiving input from a user.

In block 1302, the image analysis system determines a number of objects (e.g., a number of a plurality of objects) in a first slide image. The objects may include invasive cells, invasive cancer cells, in-situ cancer cells, lymphocytes, stroma, abnormal cells, normal cells, background cells, or any other objects or type of objects. For example, the objects may correspond to a particular object type of a plurality of object types (e.g., cancerous cells). The objects may be defined by the image analysis system and/or by a user via user input of a user computing device. For example, the user input may define a number of objects in the first slide image, a number of object in an image that includes the first slide image, etc. The number of objects in the first slide image may be a ratio, proportion, percentage, etc. of a count of the number of objects in the first slide image to a count of a number of objects in an image (e.g., the image including the first slide image). In some embodiments, the image analysis system may identify and/or obtain the first slide image (e.g., prior to determining the number of objects included in the first slide image). The image analysis system may determine a weight associated with the first slide image and the number of objects in the first slid image. The weight may specify an amount of the first slide image occupied by the objects (e.g., a percentage of the area of the first slide image occupied by the objects). In some embodiments, the image analysis system may not determine a weight. Further, the image analysis system may determine a background percentage (e.g., a portion of the first slide image that does not contain objects) and use the background percentage instead of the weight.

In some embodiments, to determine the number of objects and/or the weight, the image analysis system may obtain user input (e.g., from a user computing device) identifying or defining the number of objects and/or the weight). Further, the image analysis system may cause display, via a display and/or user interface of the user computing device, of the first slide image. The image analysis system may cause display of an interactive representation of the first slide image. Based on causing display of the first slide image, the image analysis system may obtain the user input identifying the number of objects and/or the weight.

In some embodiments, the image analysis system may obtain multiple slide images. Each slide image may be a portion of the image. Further, each slide image may include a plurality of objects. The image analysis system may determine a number of objects and a weight for each slide image. Therefore, the image analysis system can determine the number of objects in each slide image.

In block 1304, the image analysis system generates training data (e.g., training set data) based on the number of objects in the first slide image. For example, the training data may include the first slide image, object data identifying the number of objects in the first slide image, and weight data identifying the first weight. Further, the training data may include coordinates, width, height, shape information (e.g., circle, square, etc.), circumference, radius, or other information identifying the first slide image. In some embodiments, the training data may include multiple slide images, object data for each of the multiple slide images, and weight data for each of the multiple slide images. Therefore, the image analysis system can generate the training data.

In block 1306, the image analysis system trains a machine learning model (e.g., the image analysis module) to predict a number of objects in a second slide image using the training data. Based on training the machine learning model, the image analysis system may implement the machine learning model. The machine learning model may predict a number of objects in a second slide image and a second weight associated with the second plurality of objects in the second slide image. In some cases, the machine learning model may be a convolutional neural network. To predict the number of objects in a second slide image, the machine learning model may weigh the number of objects in the first slide image using the corresponding weight (e.g., to obtain a weighted average). Further, the machine learning model may outline each objects based on the weighted number of objects, count the number of outlined objects, and cause display of a number of objects in the second slide image based on the count. Therefore, the image analysis system can train and implement the machine learning model.

### Prediction of a Number of Objects Using Weights

FIG. 13B is a flow diagram showing the steps involved in training the image analysis module. FIG. 13B shows a method 1300B executed by an image analysis system, according to some examples of the disclosed technologies. The image analysis system may be similar, for example, to the image analysis system described above. It will be understood that the method 1300B may be performed by different devices (e.g., a computing device). The method 1300B may begin automatically upon receiving input from a user.

In block 1312, the image analysis system determines, for each first slide image of a plurality of first slide images, a number of objects in the first slide image. Each of the first slide images may be a portion of an image. Further, the number of objects in a particular first slide image may identify the number of objects in a particular portion of the image associated with the particular first slide image. The image analysis system may receive user input defining each portion of the image and determine the plurality of first slide images based on the user input. In some embodiments, to determine the number of objects for each slide image, the image analysis system may obtain user input (e.g., from a user computing device) identifying or defining the number of objects. Therefore, the image analysis system can determine the number of objects in each first slide image.

In block 1314, the image analysis system determines, for each first slide image of the plurality of first slide images, a weight. In some embodiments, to determine the weight for each slide image, the image analysis system may obtain user input (e.g., from a user computing device) identifying or defining the weight. Each of the weights may specify an amount of an associated first slide image occupied by the objects (e.g., a percentage of the area of the first slide image occupied by the objects). Therefore, the image analysis system can determine the weight for each first slide image.

In block 1316, the image analysis system generates training data based on, for each first slide image of the plurality of first slide images, the number of objects in the first slide image and the weight. The training data may include coordinates, width, height, shape information (e.g., circle, square, etc.), circumference, radius, or other information identifying each first slide image and the portion of the image corresponding to each first slide image. For example, the training data may include coordinates identifying a particular portion of the image and an identifier of a particular first slide image. Therefore, the image analysis system can generate the training data.

In block 1318, the image analysis system trains a first machine learning model to predict a number of objects in a second slide image using the training data. Further, the image analysis system may implement the first machine learning model and the first machine learning model may predict a number of objects in a second slide image and a number of objects in a third slide image. To predict the number of objects in a second slide image, the machine learning model may weigh the number of objects in each first slide image using the corresponding weight (e.g., to obtain a weighted average). In some embodiments, the machine learning model may not weigh the number of objects in each slide image. Further, the machine learning model may provide the number of objects in each slide image to a second machine learning model that is trained to identify weights for each slide image. Therefore, the image analysis system can train the first machine learning model.

In block 1320, in some embodiments, the image analysis system provides the output of the first machine learning model as input to a second machine learning model. The image analysis system may train the second machine learning model based on the number of objects in a slide image (e.g., the number of objects in the second slide image and the number of objects in the third slide image) as predicted by the first machine learning model. Further, the image analysis system may implement the second machine learning model. The second machine learning model may aggregate a plurality of predicted number of objects for a plurality of slide images (e.g., predictions by the first machine learning model). The second machine learning model, based on the aggregation may identify a number of objects in an image based on the predicted number of objects in each of the plurality of slides images. Therefore, the image analysis system can provide the output of the first machine learning model as input to a second machine learning model.

### CONCLUSION

The foregoing description details certain embodiments of the systems, devices, and methods disclosed herein. It will be appreciated, however, that no matter how detailed the foregoing appears in text, the systems, devices, and methods can be practiced in many ways. As is also stated above, it should be noted that the use of particular terminology when describing certain features or aspects of the disclosure should not be taken to imply that the terminology is being re-defined herein to be restricted to including any specific characteristics of the features or aspects of the technology with which that terminology is associated.

Information and signals disclosed herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logical blocks, and algorithm steps described in connection with the embodiments disclosed herein may be implemented as electronic hardware, computer software, or combinations of both. To clearly illustrate this interchangeability of hardware and software, various illustrative components, blocks, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The techniques described herein may be implemented in hardware, software, firmware, or any combination thereof. Such techniques may be implemented in any of a variety of devices such as general purposes computers, wireless communication device handsets, or integrated circuit devices having multiple uses including application in wireless communication device handsets and other devices. Any features described as devices or components may be implemented together in an integrated logic device or separately as discrete but interoperable logic devices. If implemented in software, the techniques may be realized at least in part by a computer-readable data storage medium comprising program code including instructions that, when executed, performs one or more of the methods described above. The computer-readable data storage medium may form part of a computer program product, which may include packaging materials. The computer-readable medium may comprise memory or data storage media, such as random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general purpose processor may be a microprocessor; but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein. In addition, in some aspects, the functionality described herein may be provided within dedicated software or hardware configured for encoding and decoding, or incorporated in a combined video encoder-decoder (CODEC). Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, or units are described in this disclosure to emphasize functional aspects of devices configured to perform the disclosed techniques, but do not necessarily require realization by different hardware units. Rather, as described above, various units may be combined in a codec hardware unit or provided by a collection of inter-operative hardware units, including one or more processors as described above, in conjunction with suitable software and/or firmware.

Although the foregoing has been described in connection with various different embodiments, features or elements from one embodiment may be combined with other embodiments without departing from the teachings of this disclosure. However, the combinations of features between the respective embodiments are not necessarily limited thereto. Various embodiments of the disclosure have been described. These and other embodiments are within the scope of the following claims.

1. An apparatus comprising:
a memory circuit storing computer-executable instructions; and
a hardware processing unit configured to execute the computer-executable instructions, wherein execution of the computer-executable instructions causes the hardware processing unit to:
obtain a first slide image comprising a first plurality of objects;
determine a number of the first plurality of objects in the first slide image and a first weight;
generate training set data comprising:
the first slide image,
object data identifying the number of the first plurality of objects in the first slide image, and
weight data identifying the first weight;
train a machine learning model based on the training set data; and
implement the machine learning model, wherein the machine learning model predicts a number of a second plurality of objects in a second slide image and a second weight.

2. The apparatus of claim 1, wherein the execution of the computer-executable instructions further causes the hardware processing unit to:
obtain, from memory, the first slide image; and
obtain, from a user computing device, user input identifying the number of the first plurality of objects in the first slide image.

3. The apparatus of claim 1, wherein the execution of the computer-executable instructions further causes the hardware processing unit to:
cause display, via a display of a user computing device, of the first slide image; and
obtain, from the user computing device, user input identifying the number of the first plurality of objects in the first slide image based on causing display of first slide image.

4. The apparatus of claim 1, wherein the machine learning model comprises a convolutional neural network.

5. The apparatus of claim 1, wherein the first slide image corresponds to a portion of an image, wherein the number of the first plurality of objects in the first slide image comprises a number of the first plurality of objects in the portion of the image, wherein the execution of the computer-executable instructions further causes the hardware processing unit to:
obtain, from a user computing device, user input identifying the portion of the image, wherein the training data set further comprises the portion of the image.

6. The apparatus of claim 1, wherein the number of the first plurality of objects in the first slide image comprises a number of the first plurality of objects in a portion of an image, wherein the execution of the computer-executable instructions further causes the hardware processing unit to:
obtain, from a user computing device, first user input identifying the portion of the image; and
obtain, from the user computing device, second user input identifying the number of the first plurality of objects in the first slide image.

7. The apparatus of claim 1, wherein the first slide image corresponds to a portion of an image, wherein the number of the first plurality of objects in the first slide image comprises a ratio of a count of objects in the first slide image to a count of objects in the image.

8. The apparatus of claim 1, wherein the first plurality of objects comprises at least one of:
invasive cells,
invasive cancer cells,
in-situ cancer cells,
lymphocytes,
stroma,
abnormal cells,
normal cells, or
background cells.

9. The apparatus of claim 1, wherein the execution of the computer-executable instructions further causes the hardware processing unit to:
obtain a third slide image comprising a third plurality of objects; and
determine a number of the third plurality of objects in the third slide image and a third weight, wherein the training set data further comprises:
the third slide image,
additional object data identifying the number of the third plurality of objects in the third slide image, and
additional weight data identifying the third weight.

10. The apparatus of claim 1, wherein the first slide image corresponds to a first portion of an image, wherein the execution of the computer-executable instructions further causes the hardware processing unit to:
obtain a third slide image corresponding to a second portion of the image, wherein the third slide image comprises a third plurality of objects;
determine a number of the third plurality of objects in the third slide image and a third weight, wherein the first weight is based on an amount of the first portion of the image occupied by the first plurality of objects and the third weight is based on an amount of the second portion of the image occupied by the third plurality of objects, wherein the training set data further comprises:
the third weight,
the third slide image, and
additional object data identifying the number of the third plurality of objects in the third slide image.

11. The apparatus of claim 1, wherein the machine learning model further predicts a number of a third plurality of objects in a third slide image, wherein the second slide image corresponds to a first portion of an image and the third slide image corresponds to a second portion of the image, wherein the execution of the computer-executable instructions further causes the hardware processing unit to:
train a second machine learning model based on the number of the second plurality of objects in the second slide image and the number of the third plurality of objects in the third slide image; and
implement the second machine learning model, wherein the second machine learning model aggregates a plurality of predictions for a plurality of slide images to identify a number of a plurality of objects in an image, wherein each of the plurality of predictions identifies a number of a plurality of objects in a corresponding slide image of the plurality of slide images.

12. The apparatus of claim 1, wherein the first plurality of objects correspond to a particular object type of a plurality of object types.

13. A computer-implemented method comprising:
obtaining a first slide image comprising a first plurality of objects;
determining a number of the first plurality of objects in the first slide image and a first weight;
generating training set data comprising:
the first slide image,
object data identifying the number of the first plurality of objects in the first slide image, and
weight data identifying the first weight;
training a machine learning model based on the training set data; and
implementing the machine learning model, wherein the machine learning model predicts a number of a second plurality of objects in a second slide image and a second weight.

14. A non-transitory computer-readable medium storing computer-executable instructions that, when executed by one or more computing devices, cause the one or more computing devices to:
obtain a first slide image comprising a first plurality of objects;
determine a number of the first plurality of objects in the first slide image and a first weight;
generate training set data comprising:
the first slide image,
object data identifying the number of the first plurality of objects in the first slide image, and
weight data identifying the first weight;
train a machine learning model based on the training set data; and
implement the machine learning model, wherein the machine learning model predicts a number of a second plurality of objects in a second slide image and a second weight.

15. The non-transitory computer-readable medium of claim 14, wherein execution of the computer-executable instructions by the one or more computing devices further causes the one or more computing devices to:
obtain, from a user computing device, user input identifying the number of the first plurality of objects in the first slide image.

16. The non-transitory computer-readable medium of claim 14, wherein the first slide image corresponds to a portion of an image, wherein the number of the first plurality of objects in the first slide image comprises a percentage of the number of the first plurality of objects in the first slide image as compared to a number of a plurality of objects in the image.

17. The non-transitory computer-readable medium of claim 14, wherein the first plurality of objects comprises at least one of:
invasive cells,
invasive cancer cells,
in-situ cancer cells,
lymphocytes,
stroma,
abnormal cells,
normal cells, or
background cells.

18. The non-transitory computer-readable medium of claim 14, wherein execution of the computer-executable instructions by the one or more computing devices further causes the one or more computing devices to:
obtain a third slide image comprising a third plurality of objects; and
determine a number of the third plurality of objects in the third slide image and a third weight, wherein the training set data further comprises:
the third slide image,
additional object data identifying the number of the third plurality of objects in the third slide image, and
additional weight data identifying the third weight.

19. The non-transitory computer-readable medium of claim 14, wherein the first slide image corresponds to a first portion of an image, wherein the first weight is based on an amount of the first portion of the image occupied by the first plurality of objects.

20. The non-transitory computer-readable medium of claim 14, wherein the image, wherein the second slide image corresponds to a first portion of an image and the third slide image corresponds to a second portion of the image, wherein execution of the computer-executable instructions by the one or more computing devices further causes the one or more computing devices to:
train a second machine learning model based on the number of the second plurality of objects in the second slide image and the number of the third plurality of objects in the third slide image; and
implement the second machine learning model, wherein the second machine learning model aggregates a plurality of predictions for a plurality of slide images to identify a number of a plurality of objects in an image, wherein each of the plurality of predictions identifies a number of a plurality of objects in a corresponding slide image of the plurality of slide images.

21.-22. (canceled)
